(19) United States
(12) Patent Application Publication
(10) Pub. No.: US 2021/0261510 A1
(43) Pub. Date: Aug. 26, 2021

(54) Ionic Liquid Preparation

(71) Applicant: Seren Technologies Limited, Redcar, Cleveland (GB)

(72) Inventors: Peter Nockemann, Belfast (GB); Donnacha Brolly, Cleveland (GB); Ena Bradley, Cleveland (GB); Eadaoin Mccourt, Cleveland (GB)

(21) Appl. No.: 17/252,255

(22) PCT Filed: Jun. 14, 2019
(86) PCT No.: PCT/GB2019/051660

§ 371 Date: Dec. 14, 2020

(30) Foreign Application Priority Data
Jun. 15, 2018 (GB) 1809813.7

(51) Int. Cl. C07D 233/61; C07F 9/54; C22B 59/00; C22B 3/36

(52) U.S. Cl. CPC C07D 233/61; C07F 9/5442; C22B 3/06; C22B 3/36; C22B 59/00

(57) ABSTRACT

A process for preparing a cationic species [Cat+] for an ionic liquid, said process comprising reacting a reagent (1) H 2 N-L-[Z] with a reagent (2) LG-L 2-EDG, to form a cationic species EDG-L 2-[Z+]-L-N(L 2-EDG) 2, wherein the process is carried out in a sealed reactor at a temperature of at least 100° C.

The present invention relates to methods for preparing cations for use in ionic liquids. The ionic liquids are specifically designed for use in the extraction and separation of rare earth metals.

Rare earth metals, which include the lanthanides (La to Lu), Y, and Sc, have unique physicochemical properties which make them crucial components of numerous high-tech products and environmental technologies such as wind mills, LCD/LED displays, phosphors, magnet drives (hard disk), and others. These applications demand a continuous supply of high purity rare earth metals to the industries, which is currently met by mining and processing the natural ores of these metals. However, there are concerns that the exponentially increasing demand of these metals will surpass the supply in coming years and therefore, it has become attractive to explore other secondary sources of these valuable metals. One such source is the recovery of rare earth metals from end-of-life and manufacturing wastes materials (often referred to as “urban mining”), which, though quite challenging, can potentially provide a continuous supply of the rare earth metals. One of most important requirements of urban mining is the development of cost effective and robust separation processes/technologies which allow selective and efficient separation of rare earth metals from each other (intra-group separation) to provide high purity rare earth metals.

During the last five decades various processes such as liquid-liquid extraction (e.g. Rhône-Poulenc process), ion exchange, and precipitation have been developed. Among the various available technologies, liquid-liquid extraction has been found to be the most suitable commercial process owing to its scalability, adaptability, and recyclability. Additionally, the liquid-liquid extraction processes used to date employ commercial organophosphorus extractants which do not possess specific selectivity for individual rare earth metals, thereby leading to a number of stages to separate rare earth metals from each other (see Table 1). Furthermore, additional processing steps are generally required to recover the rare earth metal in high purity. These factors lead to manifold increase in processing costs thereby putting strain on overall costing of consumer products. Also, most employed methods for the separation of rare earth metals necessitate the use of organic solvents, which due to their toxicity, volatility and flammability are not considered environmentally friendly.

Some of the currently used industrial liquid-liquid extraction processes available for intra-group separation of rare earth metals (e.g. separation of dysprosium from neodymium) are compared in Table 1.

The separation factor for an individual rare earth metal pair is expressed as the ratio of the distribution ratios (D_{M}) of the rare earth metals, where the distribution ratio of an individual rare earth metal is determined as the ratio of its concentration in the non-aqueous phase to that in the aqueous phase i.e. D_{M}=[M]_{N-Aq}/[M]_{Aq}. For example, the separation factor of Dysprosium with respect to Neodymium=D_{Dy}/D_{Nd}.

TABLE 1
Comparison of the separation factors of commonly used
REM extractants.

| Liquid- | | Sepa- | |
|---|---|---|---|
| liquid | | ration | |
| extraction | Major component | factor | Reference |
| HDEHP | Bis-(2-ethylhexyl)- | 41.5 | C. K. Gupta, N. |
| process | phosphoric acid | (Dy/Nd) | Krishnamurthy, Extractive |
| | | | Metallurgy of Rare |
| | | | Earths, CRC, New York, 2005, |
| | | | pp. 1-484. |
| Cyanex | Bis-(2,4,4- | 1.36 | B. Swain, E.O. Otu, |
| 272 | trimethylpentyl) | (Dy/Nd) | Separation and Purification |
| process | phosphinic acid | | Technology, 83, (2011), 82-90 |
| Cyanex | Bis-(2,4,4- | 239.3 | M. Yuan, A. Luo, D. Li, Acta |
| 302 | trimethylpenty1)- | (Dy/Nd) | Metall. Sin. 1995, 8, 10-14. |
| process | monothiophosphinic | | |
| | acid | | |
| Synergist | 2-ethylhexylphos- | 1.17 | N. Song, S. Tong, W. Liu, |
| process | phonic acid mono- | (Dy/Nd) | Q.Jia, W.Zhoua and W.Liaob, |
| | (2-ethylhexyl) | | J. Chem. Technol. |
| | ester; sec-nonyl- | | Biotechnol., 2009, 84, 1798- |
| | phenoxy acetic acid | | 1802. |

Another of the most commonly used organophosphorous extractants, P507 (2-ethylhexyl phosphoric acid mono(2-ethylhexyl) ester), also gives low separation factors, with the selectivity for heavy rare earth metals generally being lower than for light rare earth metals (e.g. Tm/Er (3.34), Yb/Tm (3.56), and Lu/Yb (1.78)). Another significant deficiency of many common rare earth metal extractants such as P507 is that it is difficult to strip heavy rare earth metals completely, especially for Tm(III), Yb(III), and Lu(III), even at higher acidity. Low selectivity for rare earth metals results in too many stages required for effective separation, the low extractability of rare earth metals demanding the use of higher concentrations of the extractant. The production of organophosphorous extractants also requires complicated synthetic procedures starting from hazardous starting materials and the stability and recyclability of these extractants is limited. Emulsification and leaching of extractants has been identified as another common problem.

A chelating diamide extractant attached to a silica support was reported by Fryxell et al. for the separation of lanthanides (Inorganic Chemistry Communications, 2011, 14, 971-974).

However, this system was unable to extract rare earth metals under acidic conditions (pH<5) and crucially showed very low uptake and separation factors between rare earth metals.

Ionic liquids have also been used as potential extractants for rare earth metals. Binnemans et al. reported the extraction of Nd and Dy or Y and Eu from mixtures of transition metal compounds with a betainium bis(trifluoromethyl-sulfonyl)imide ionic liquid (Green Chemistry, 2015, 17, 2150-2163; Green Chemistry, 2015, 17, 856-868). However, this system was unable to selectively perform intra-group separation between rare earth metals.

Chai et al. reported the use of an ionic liquid based on 2-ethylhexyl phosphonic acid mono(2-ethylhexyl) ester (P507) with a trioctylmethylammonium cation for separation of rare earth metals (Hydrometallurgy, 2015, 157(C), 256-260). In this case only low distribution factors and separation factors were observed, indicating a lack of extractability and selectivity. In addition, during recovery of the rare earth metal from the ionic liquid, the acid added will decompose the acid-base pair ionic liquid, which must then be regenerated by metathesis.

Separation of Nd and Dy was reported by Schelter et al., whereby separation was achieved by precipitation using a tripodal nitroxide ligand to form Nd and Dy complexes with differing solubilities in benzene. However, precipitation is not considered to be a commercially viable process and, in addition, the process requires the use of specific rare earth metal precursors and an inert, moisture-free environment, which is highly impractical for commercial scale up.

This method also relies on the use of benzene to achieve high separation, which is a very toxic solvent.

Therefore, there is a need for the development of effective processes that enhance separation selectivity and extractability, whilst minimizing environmental pollution.

By using an ionic liquid having a cation comprising particular features, it has been found that rare earth metals may be extracted and separated from each other with increased selectivity and extractability in comparison to known methods using different extractants. As the method uses an ionic liquid, the extractant can also provide decreased volatility and flammability, potentially leading to safer and more environmentally friendly rare earth metal extraction.

A suitable ionic liquid has the formula [Cat⁺][X⁻] in which:
[Cat⁺] represents a cationic species having the structure:

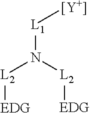

where: [Y⁺] comprises a group selected from ammonium, benzimidazolium, benzofurani um, benzothiophenium, benzotriazoli um, borolium, cinnolinium, diazabicyclodecenium, diazabicyclononenium, 1,4-diazabicyclo[2.2.2]octanium, diazabicyclo-undecenium, dithiazolium, furanium, guanidinium, imidazolium, indazolium, indolinium, indolium, morpholinium, oxaborolium, oxaphospholium, oxazinium, oxazolium, iso-oxazolium, oxothiazolium, phospholium, phosphonium, phthalazinium, piperazinium, piperidinium, pyranium, pyrazinium, pyrazolium, pyridazinium, pyridinium, pyrimidinium, pyrrolidinium, pyrrolium, quinazolinium, quinolinium, iso-quinolinium, quinoxalinium, quinuclidinium, selenazolium, sulfonium, tetrazolium, thiadiazolium, iso-thiadiazolium, thiazinium, thiazolium, iso-thiazolium, thiophenium, thiuronium, triazinium, triazolium, iso-triazolium and uronium groups;
   each EDG represents an electron donating group; and
   L₁ represents a linking group selected from C₁₋₁₀ alkanediyl, C₂₋₁₀ alkenediyl, C₁₋₁₀ dialkanylether and C₁₋₁₀ dialkanylketone groups;
   each L₂ represents a linking group independently selected from C₁₋₂ alkanediyl, C₂ alkenediyl, C₁₋₂ dialkanylether and C₁₋₂ dialkanylketone groups; and
[X³¹ ] represents an anionic species.

Accordingly, there is a need for effective and efficient methods for preparing these ionic liquids, in particular the rare earth metal-binding cation of these ionic liquids.

Thus, in a first aspect, the present invention provides a process for preparing a cationic species [Cat⁺] as defined above, said process comprising carrying out the following reaction:

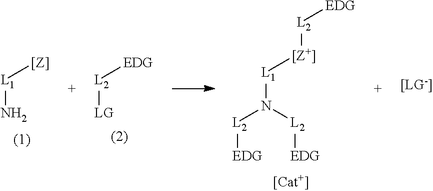

where: LG represents a leaving group;

wherein the process is carried out in a sealed reactor at a temperature of greater than 100° C.

It has surprisingly been found that, by carrying out the reaction under these conditions, the cationic species may be readily prepared in high yields in just a short period of time. Thus, in preferred embodiments, the reaction between reagents (1) and (2) may be carried out for a period of up to 24 hours, preferably up to 12 hours, and more preferably up to 6 hours. The reaction may be carried out for a period of at least 0.5 hours, preferably at least 1 hour, and more preferably at least 2 hours. Thus, the reaction may be carried out for a period of from 0.5 to 24 hours, preferably from 1 to 12 hours and more preferably from 2 to 6 hours.

Preferably, the process is carried out at a temperature of at least 115° C., and more preferably at least 125° C. The process may be carried out at a temperature of up to 180° C., preferably up to 170° C., and more preferably up to 145° C. Thus, the process may be carried out at a temperature from 100 to 200° C., preferably from 115 to 180° C., and more preferably from 125 to 160° C. In some instances, these temperatures are above the boiling point of one or more of the reagents and solvent.

The reaction is carried out in a sealed reactor. Suitable reactors include pressure vessels, such as a sealed tube reactor. By using a combination of elevated temperature and a sealed vessel, slight pressure may built up in the reactor which may aid the progress of the reaction.

Thus, in some embodiments, the reaction may be carried out under pressure. Preferably, the pressure is generated by the reaction itself, rather than by an external source. For instance, the reaction may be carried out at a pressure of at least 105 kPa, preferably at least 200 kPa, and more preferably at least 250 kPa. The process may be carried out at a pressure of up to 500 kPa, preferably up to 400 kPa, and more preferably up to 350 kPa. Thus, the process may be carried out at a pressure of from 105 to 500 kPa, preferably from 200 to 400 kPa, and more preferably from 250 to 350 kPa.

The reaction mixture will typically be mixed. Any suitable apparatus may be used to achieve this and mixing apparatuses are well known in the art. For example, the mixture may be mixed using an agitator or stirrer.

Reagent (2) may be used in an amount of at least 1 molar equivalent, preferably at least 2 molar equivalents and more preferably at least 2.5 molar equivalents as compared to reagent (1). Reagent (2) may be used in an amount of up to 6 molar equivalents, preferably up to 4 molar equivalents, and more preferably up to 3.5 molar equivalents as compared to reagent (1). Thus, reagent (2) may be used in an amount of from 1 to 6 molar equivalents, preferably from 2 to 4 molar equivalents and more preferably from 2.5 to 3.5 molar equivalents as compared to reagent (1).

The reaction is preferably carried out in the presence of a base. The base may be a nitrogen-containing base such as a tertiary amine-containing base or pyridine. Preferably the base is liquid under ambient conditions (e.g. at a temperature of 20° C. and a pressure of 100 kPa). Particularly preferred bases are trialkylamines, e.g. where each alkyl group is independently selected from C₁₋₆ alkyl groups, such as triethylamine or diisopropylethylamine.

The base may be used in an amount of at least 1 molar equivalent, preferably at least 2 molar equivalents and more preferably at least 3 molar equivalents as compared to reagent (1). The base may be used in an amount of up to 10 molar equivalents, preferably up to 8 molar equivalents and more preferably up to 5 molar equivalents as compared to reagent (1). Thus, the base may be used in an amount of from 1 to 10 molar equivalents, preferably from 2 to 8 molar equivalents and more preferably from 3 to 5 molar equivalents as compared to reagent (1). Without wishing to be bound by theory, it is believed that the use of a large excess of base is desirable as it ‘mops up’ the [H⁺][LG⁻] that forms during the reaction and which may interfere with the reaction progress. For instance, where LG=CI, the excess of base mops of HCl which might otherwise interfere with the reaction.

The reaction may be carried out in the presence of an organic solvent. Preferred organic solvents include halogenated solvents, e.g. dichloromethane or trichloromethane, and substituted benzene compounds, e.g. toluene. Chloroform gives particularly good yields in a short amount of time.

During the addition reaction between reagents (1) and (2), a leaving group LG is lost from reagent (2) and the cation is produced. The cation is produced in the form of an ionic liquid in which the anion is the leaving group. In other words, that cation is prepared in the form of an ionic liquid having the formula [Cat⁺][LG⁻].

A “leaving group” as used herein will be understood to mean a group that may be displaced from a molecule by reaction with a nucleophilic centre, in particular a leaving group will depart with a pair of electrons in heterolytic bond cleavage. A leaving group is usually one that is able to stabilize the additional electron density that results from bond heterolysis. Such groups are well-known in the field of chemistry.

It will be appreciated that a leaving group as defined herein will be such that the primary amine coupled by L₁ to [Z] may displace the leaving group to form a bond between the nitrogen and an L₂ group.

Leaving groups may, for example, include a group selected from —OSO₂CF₃ (i.e. —OTf), —SO₂R such as tosylate (—OTs) or mesylate (—OMs), halides (such as —Cl, —Br and —I), —OR, —OR₂⁺, —ONO₂, —PO(OR)₂, —N₂⁺, —SR₂⁺, and —NR₃⁺, where R is selected from H, C₁₋₆ alkyl and C₄₋₁₀ aryl groups. Preferably, the leaving group -LG is selected from —OTf, —OSO₂R, and halides. Halides are particularly preferred as leaving groups, in particular —Cl.

Once the reaction is complete, it may be allowed to cool to room temperature, e.g. to a temperature of from 15 to 30° C. The reaction mixture may then be filtered to remove any solids that may be present, such as salts (e.g. trimethylamine hydrochloride salt) that form as a by-product during the reaction. However, in some embodiments, particularly where an excess of base is used, it is not necessary to filter the reaction mixture.

The cation, [Cat⁺], may be washed to remove any impurities that are present in the ionic liquid [Cat⁺][LG³¹ ]. The cation may be washed more than once, preferably more than three times, and more preferably more than five times. The ionic liquids that are produced using a process of the present invention are typically immiscible with water. Thus, water or aqueous solutions are particularly suitable for washing the cation, since they may form a separate aqueous phase. In embodiments, the process further comprises washing the cation with water or an aqueous solution, e.g. until the aqueous phase is neutral, i.e. has a pH of about 7 (e.g. 6.5 to 7.5).

The cation may be washed with just water, however it is generally preferred for the cation to be washed with an acid, then a base, then with water. For instance, the cation may be washed with acid until the aqueous phase is mildly acidic, e.g. 2≤pH≤6. The cation may then be washed with a base until the aqueous phase is less acidic, e.g. 8≤pH≤9. Finally, the cation may be washed with water until the aqueous phase is neutral, i.e. a pH of about 7.

The acid and base wash solutions may have a molarity of at least 0.05 M, preferably at least 0.1 M, and more preferably at least 0.5 M. The wash solutions may have a molarity of up to 3 M, preferably up to 2 M, and more preferably up to 1.5 M. Thus, the wash solutions may have a molarity of from 0.05 to 3 M, preferably from 0.1 to 2 M, and more preferably from 0.5 to 1.5 M.

Suitable acids for the acid wash solution include protic acids such as hydrogen halides (e.g. HBr, HCl, HI), sulfuric acid, phosphoric acid and acetic acid. Since the cation is prepared in the form of an ionic liquid having the formula [Cat⁺][LG³¹ ], some anion exchange may take place between the ionic liquid and the acid. It is therefore generally preferred that the anion of the acid is the same as the leaving group.

Suitable bases for the base wash solution include carbonates (e.g. Na₂CO₃), though a wide range of other basis may also be used.

The solvent that is used for the reaction may be removed from the cation under vacuum to provide the cation as an isolated ionic liquid [Cat⁺][LG³¹ ]. Where the cation is purified by washing, the solvent that is used to carry out the reaction is preferably removed from the purified cation, i.e. after washing.

In a further aspect, the present invention provides a process for preparing an ionic liquid having the formula [Cat⁺][X³¹ ], said process comprising: preparing an ionic liquid having the formula [Cat⁺][LG³¹ ] using a process described herein; and where LG⁻ is not the same as X⁻, carrying out the following anion exchange reaction:

[Cat⁺][LG³¹ ]+[X⁻]→[Cat⁺][X⁻]+[LG⁻]

Where [LG⁻] is the same as [X⁻], i.e. the cation is prepared in the form of an anionic liquid having the formula [Cat⁺][X⁻], then it is not necessary to carry out an anion exchange reaction. However, where the target ionic liquid has a different anion from that present in [Cat⁺][LG³¹ ], then it is necessary to carry out an anion exchange reaction with anion [X⁻].

Anion [X⁻] will typically be used in the form of a salt in which the cation is a metal. The metal may be Group 1 metal, such as lithium, sodium or potassium, or a Group 2 metal, such as magnesium or calcium. Sodium is particularly preferred.

The anion [X⁻] may be used in an amount of at least 1 molar equivalent, preferably at least 1.05 molar equivalents, and more preferably at least 1.1 molar equivalents as compared to the leaving group anion in [Cat⁺][LG³¹ ]. The anion may be used in an amount of up to 2.5 molar equivalents, preferably up to 2 molar equivalents, and more preferably up to 1.5 molar equivalents as compared to the leaving group anion in [Cat⁺][LG³¹ ]. The anion may be used in an amount of from 1 to 2.5 molar equivalents, preferably from 1.05 to 2 molar equivalents, and more preferably from 1.1 to 1.5 molar equivalents as compared to the leaving group anion in [Cat⁺][LG³¹ ]. Use of a slight excess of the anion [X⁻] encourages high levels of anion exchange.

The anion exchange reaction may be carried out at room temperature (e.g. to a temperature of from 15 to 30° C.). For instance, the reaction may be carried out without the application of heat or cooling.

The anion exchange reaction may be carried out at ambient pressure (e.g. at a pressure of approximately 100 kPa). For instance, the reaction may be carried out without the application of pressure.

The anion-exchange reaction may be carried out for a period of at least 0.1 hours, preferably at least 0.25 hours, and more preferably at least 0.5 hours. The anion-exchange reaction may be carried out for a period of up to 5 hours, preferably up to 3 hours, and more preferably up to 2 hours. The anion-exchange reaction may be carried out for a period of from 0.1 to 5 hours, preferably from 0.25 to 3 hours, and more preferably from 0.5 to 2 hours.

The reaction may be carried out in the presence of an organic solvent. Preferred organic solvents include halogenated solvents, e.g. dichloromethane or trichloromethane, and non-polar solvents, e.g. toluene, benzene, pentane, hexane, cyclohexane and the like. Trichloromethane is particularly preferred.

The reaction mixture will typically be mixed. Any suitable apparatus may be used to achieve this and mixing apparatuses are well known in the art. For example, the mixture may be mixed using an agitator or stirrer.

The process of preparing the ionic liquid [Cat⁺][X⁻] may further comprise washing the ionic liquid once the reaction has finished. In some embodiments, the ionic liquid may be washed more than once, preferably more than twice, such as more than three times. The ionic liquid is preferably washed with water, e.g. until the aqueous phase is neutral, i.e. has a pH of about 7 (e.g. 6.5 to 7.5).

The solvent that is used for the anion exchange reaction may be removed from the ionic liquid [Cat⁺][X⁻] under vacuum to provide the ionic liquid in an isolated form. Where the ionic liquid is purified by washing, the solvent that is used to carry out the anion exchange reaction is preferably removed from the purified ionic liquid, i.e. after washing.

One of the main advantages underlying the invention is that process of the present invention produces the ionic liquid [Cat⁺][X⁻] at an unexpectedly high yield. For instance, the ionic liquid may be produced at a yield of greater than 50%, preferably greater than 60%, and more preferably greater than 70%. This yield is obtainable even after the ionic liquid has been purified by washing and isolated.

The term “ionic liquid” as used herein refers to a liquid that is capable of being produced by melting a salt, and when so produced consists solely of ions. An ionic liquid may be formed from a homogeneous substance comprising one species of cation and one species of anion, or it can be composed of more than one species of cation and/or more than one species of anion. Thus, an ionic liquid may be composed of more than one species of cation and one species of anion. An ionic liquid may further be composed of one species of cation, and one or more species of anion. Still further, an ionic liquid may be composed of more than one species of cation and more than one species of anion.

The term “ionic liquid” includes compounds having both high melting points and compounds having low melting points, e.g. at or below room temperature. Thus, many ionic liquids have melting points below 200° C., particularly below 100° C., around room temperature (15 to 30° C.), or even below 0° C. Ionic liquids having melting points below around 30° C. are commonly referred to as “room temperature ionic liquids” and are often derived from organic salts having nitrogen-containing heterocyclic cations. In room temperature ionic liquids, the structures of the cation and anion prevent the formation of an ordered crystalline structure and therefore the salt is liquid at room temperature.

Ionic liquids are most widely known as solvents. Many ionic liquids have been shown to have negligible vapour pressure, temperature stability, low flammability and recyclability. Due to the vast number of anion/cation combinations that are available it is possible to fine-tune the physical properties of the ionic liquid (e.g. melting point, density, viscosity, and miscibility with water or organic solvents) to suit the requirements of a particular application.

The ionic liquids prepared according to the present invention have the formula [Cat⁺][X⁻].

The cationic species [Cat⁺] has the structure:

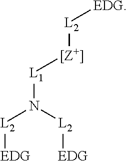

L₁ represents a linking group selected from C₁₋₁₀ alkanediyl, C₂₋₁₀ alkenediyl, C₁₋₁₀ dialkanylether and C₁₋₁₀ dialkanylketone groups.

In preferred embodiments, L₁ represents a linking group selected from C₁₋₁₀ alkanediyl and C₁₋₁₀ alkenediyl groups, more preferably selected from C₁₋₅ alkanediyl and C₂₋₅ alkenediyl groups, and most preferably selected from C₁₋₅ alkanediyl groups, for example a linking group selected from —CH₂—, —C₂H₄— and —C₃H₆—.

Each L₂ also represents a linking group. Each L₂ is independently selected from C₁₋₂ alkanediyl, C₂ alkenediyl, C₁₋₂ dialkanylether and C₁₋₂ dialkanylketone groups

In preferred embodiments, each L₂ represents a linking group independently selected from C₁₋₂ alkanediyl and C₂ alkenediyl groups, preferably selected from C₁₋₂ alkanediyl groups, for example independently selected from —CH₂— and —C₂H₄—.

Each EDG represents an electron donating group. The term electron donating group (EDG) as used herein will be understood to include any group having a pair of electrons available to form a coordinate bond with an acceptor. In particular, it will be appreciated that an electron donating group, as defined herein, refers to groups having an available pair of electrons able to coordinate to a rare earth metal to form a metal-ligand complex. It will also be understood that the EDGs will typically have a single atom from which the electrons are donated to form a bond. However, electrons may alternatively be donated from one or more bonds between atoms, i.e. EDG may represent a ligand with a hapticity of 2 or more.

Each EDG may be any suitable electron donating group able to form a coordinate bond with a rare earth metal to form a metal-ligand complex.

Preferably, each EDG represents an electron donating group independently selected from —CO₂R^{x}, —OC(O)R^{x}, —CS₂R^{x}, —SC(S)R^{x}, —S(O)OR^{x}, —OS(O)R^{x}, —NR^{x}C(O)NR^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)N R^{y}R^{z}, C(O)NR^{y}R^{z}, C(S)NR^{y}R^{z}, wherein R^{x}, R^{y} and R^{z} are independently selected from H or C₁₋₆ alkyl. More preferably, each EDG represents an electron donating group independently selected from —CO₂R^{x} and —C(O)NR^{y}R^{z}, wherein R^{x}, R^{y} and R^{z} are each independently selected from C₃₋₆ alkyl.

In preferred embodiments, each -L₂-EDG represents an electron donating group independently selected from:

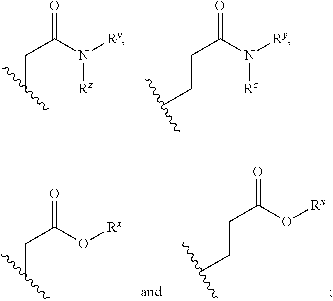

wherein R^{y}═R^{z}, and wherein R^{x}, R^{y} and R^{z} are each selected from C₃₋₆ alkyl, preferably C₄ alkyl, for example i-Bu.

More preferably, each -L₂-EDG represents an electron donating group independently selected from:

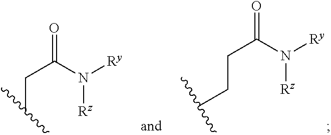

wherein R^{y}═R^{z}, and wherein R^{y} and R^{z} are selected from C₃₋₆ alkyl, preferably C₄ alkyl, for example i-Bu.

[Z⁺] represents a group selected from ammonium, benzimidazolium, benzofuranium, benzothiophenium, benzotriazolium, borolium, cinnolinium, diazabicyclodecenium, diazabicyclononenium, 1,4-diazabicyclo[2.2.2]octanium, diazabicyclo-undecenium, dithiazolium, furanium, guanidinium, imidazolium, indazolium, indolinium, indolium, morpholinium, oxaborolium, oxaphospholium, oxazinium, oxazolium, iso-oxazolium, oxothiazolium, phospholium, phosphonium, phthalazinium, piperazinium, piperidinium, pyranium, pyrazinium, pyrazolium, pyridazinium, pyridinium, pyrimidinium, pyrrolidinium, pyrrolium, quinazolinium, quinolinium, iso-quinolinium, quinoxalinium, quinuclidinium, selenazolium, sulfonium, tetrazolium, thiadiazolium, iso-thiadiazolium, thiazinium, thiazolium, iso-thiazolium, thiophenium, thiuronium, triazinium, triazolium, iso-triazolium and uronium groups.

In preferred embodiments, [Z⁺] represents an acyclic cation selected from:

[—N(R^{a})(R^{b})—]⁺, [—P(R^{a})(R^{b})—]⁺ and [—S(R^{a})—]⁺,
wherein: R^{a} and R^{b} are each independently selected from optionally substituted C₁₋₃₀ alkyl, C₃₋₈ cycloalkyl and C₆₋₁₀ aryl groups.

In other preferred embodiments, [Z⁺] represents a cyclic cation selected from:

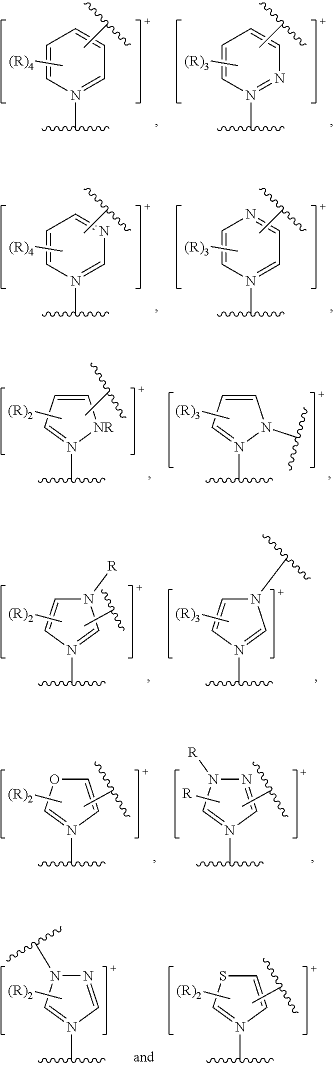

wherein: each R group is independently selected from: hydrogen and optionally substituted C₁₋₃₀ alkyl, C₃₋₈ cycloalkyl and C₆₋₁₀ aryl groups, or any two R groups attached to adjacent carbon atoms form an optionally substituted methylene chain —(CH₂)_{q}— where q is from 3 to 6.

In preferred embodiments, each R group is independently selected from H and unsubstituted C₁₋₅ alkyl groups. More preferably, each R group is H.

In particularly preferred embodiments, [Z⁺] represents a cyclic cation selected from:

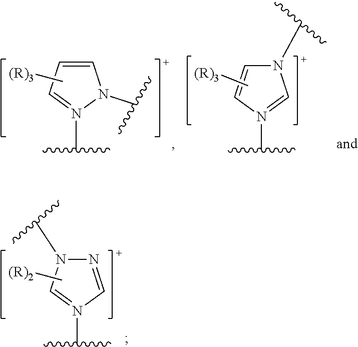

and most preferably [Z⁺] represents the cyclic cation:

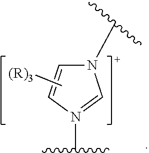

In another preferred embodiment of the invention, [Z⁺] represents a saturated heterocyclic cation selected from cyclic ammonium, 1,4-diazabicyclo[2.2.2]octanium, morpholinium, cyclic phosphonium, piperazinium, piperidinium, quinuclidinium, and cyclic sulfonium.

Preferably, [Z⁺] represents a saturated heterocyclic cation having the formula:

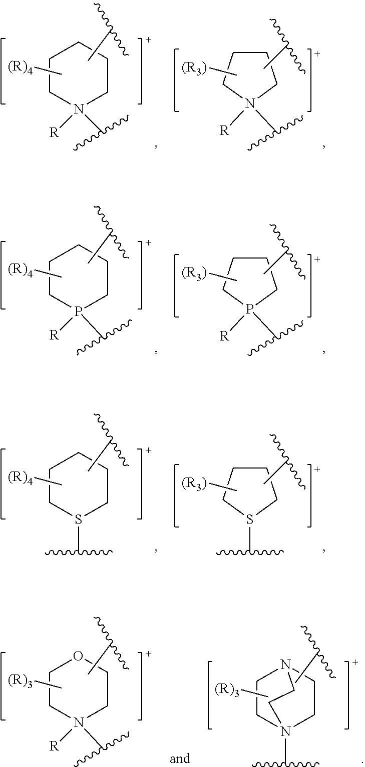

wherein: each R group is as defined above.

It will be appreciated that, as set out in detail herein, the extraction of rare earth metals is provided by the specific functionality of the cation of the ionic liquid. Thus, any suitable anionic species [X⁻] may be used as part of the ionic liquid described herein.

Preferably, [X⁻] represents one or more anionic species selected from: hydroxides, halides, perhalides, pseudohalides, sulphates, sulphites, sulfonates, sulfonimides, phosphates, phosphites, phosphonates, phosphinates, methides, borates, carboxylates, azolates, carbonates, carbamates, thiophosphates, thiocarboxylates, thiocarbamates, thiocarbonates, xanthates, thiosulfonates, thiosulfates, nitrate, nitrite, tetrafluoroborate, hexafluorophosphate and perchlorate, halometallates, amino acids, borates, polyfluoroalkoxyaluminates.

For example, [X⁻] preferably represents one or more anionic species selected from:
a) a halide anion selected from: F⁻, or, Br, 1⁻;
b) a perhalide anion selected from: [I₃]⁻, [I₂Br]⁻, [IBr₂]⁻, [Br₃]⁻, [Br₂C]⁻, [BrCl₂]⁻, [ICl₂]⁻, [I₂Cl]⁻, [Cl₃]⁻;
c) a pseudohalide anion selected from: [N₃]⁻, [NCS]⁻, [NCSe]⁻, [NCO]⁻, [CN]⁻;
d) a sulphate anion selected from: [HSO₄]⁻, [SO₄]²⁻, [R²OSO₂O]⁻;
e) a sulphite anion selected from: [HSO₃]⁻, [SO₃]²⁻, [R²OSO₂];
f) a sulfonate anion selected from: [R¹SO₂O]⁻;
g) a sulfonimide anion selected from: [(R¹SO₂)₂N]⁻;
h) a phosphate anion selected from: [H₂PO₄]⁻, [HPO₄]²⁻, [PO₄]³⁻, [R²OPO₃]²⁻, [(R²O)₂PO₂]⁻;
i) a phosphite anion selected from: [H₂PO₃]⁻, [HPO₃]²⁻, [R²OPO₂]²⁻, [(R²O)₂PO]⁻;
j) a phosphonate anion selected from: [R¹PO₃]²⁻, [R¹P(O)(OR²)O]⁻;
k) a phosphinate anion selected from: [R¹R²P(O)O]⁻;
l) a methide anion selected from: [(R¹SO₂)₃C]⁻;
m) a borate anion selected from: [bisoxalatoborate], [bismalonatoborate] tetrakis[3,5-bis(trifluoromethyl)phenyl]borate, tetrakis(pentafluorophenyl)borate;
n) a carboxylate anion selected from: [R²CO₂]⁻;
o) an azolate anion selected from: [3,5-dinitro-1,2,4-triazolate], [4-nitro-1,2,3-triazolate], [2,4-dinitroimidazolate], [4,5-dinitroimidazolate], [4,5-dicyano-imidazolate], [4-nitroimidazolate], [tetrazolate];
p) a sulfur-containing anion selected from: thiocarbonates (e.g. [R²OCS₂]⁻, thiocarbamates (e.g. [R²₂NCS₂]⁻), thiocarboxylates (e.g. [R¹CS₂]⁻), thiophosphates (e.g. [(R²O)₂PS₂]⁻), thiosulfonates (e.g. [RS(O)₂S]⁻), thiosulfates (e.g. [ROS(O)₂S]⁻);
q) a nitrate ([NO₃]⁻) or nitrite ([NO₂]⁻) anion;
r) a tetrafluoroborate ([BF₄⁻]), hexafluorophosphate ([PF₆⁻]), hexfluoroantimonate ([SbF₆⁻]) or perchlorate ([CIO₄⁻]) anion;
s) a carbonate anion selected from [CO₃]²⁻, [HCO₃]⁻, [R²CO₃]⁻; preferably [MeCO₃]⁻;
t) polyfluoroalkoxyaluminate anions selected from [Al(OR^{F})₄⁻], wherein R^{F} is selected from C₁₋₆ alkyl substituted by one or more fluoro groups;
where: R¹ and R² are independently selected from the group consisting of C₁₋₁₀ alkyl, C₆ aryl, C₁₋₁₀ alkyl(C₆)aryl and C₆ aryl(C₁₋₁₀)alkyl each of which may be substituted by one or more groups selected from: fluoro, chloro, bromo, iodo, C₁₋₆ alkoxy, C₂₋₁₂ alkoxyalkoxy, cycloalkyl, C₆₋₁₀ aryl, C₇₋₁₀ alkaryl, C₇₋₁₀ aralkyl, —CN, —OH, —SH, —NO₂, —CO₂R^{x}, —OC(O)R^{x}, —C(O)R^{x}, —C(S)R^{x}, —CS₂R^{x}, —SC(S)R^{x}, —S(O)(C₁₋₆)alkyl, —S(O)O(C₁₋₆)alkyl, —OS(O)(C₁₋₆)alkyl, —S(C₁₋₆)alkyl, —S—S(C₁₋₆ alkyl), —NR^{x}C(O)NR^{y}R^{z}, —NR^{x}C(O)OR^{y}, —OC(O)NR^{y}R^{z}, —N R^{x}C(S)OR^{y}, —OC(S)NR^{y}R^{z}, —NR^{x}C(S)SR^{y}, —SC(S)NR^{y}R^{z}, —NR^{x}C(S)NR^{y}R^{z}, —C(O)NR^{y}R^{z}, —C(S)NR^{y}R^{z}, —NR^{y}R^{z}, or a heterocyclic group, wherein R^{x}, R^{y} and R^{z} are independently selected from hydrogen or C₁₋₆ alkyl, wherein R¹ may also be fluorine, chlorine, bromine or iodine.

While [X⁻] may be any suitable anion, it is preferred that [X⁻] represents a non-coordinating anion. The term “non-coordinating anion” used herein, which is common in the field of ionic liquids and metal coordination chemistry, is intended to mean an anion that does not coordinate with a metal atom or ion, or does so only weakly. Typically, non-coordinating anions have their charge dispersed over several atoms in the molecule which significantly limits their coordinating capacity. This limits the effect interference of the anion with the selective coordination of the cation [Cat⁺] with the rare earth metal.

Thus, preferably, [X⁻] represents one or more non-coordinating anionic species selected from: bistriflimide, triflate, bis(alkyl)phosphinates such as bis(2,4,4-trimethylpentyl)phosphinate, tosylate, perchlorate, [Al(OC(CF₃)₃)₄⁻], tetrakis[3,5-bis(trifluoromethyl)phenyl]borate, tetrakis(pentafluorophenyl)borate, tetrafluoroborate, hexfluoroantimonate and hexafluorophosphate anions; and most preferably from bistriflimide, triflate and and bis(2,4,4-trimethylpentyl)phosphinate anions. Phosphinate anions in particular have been shown to give high levels of extractability.

The present invention further provides a method for extracting a rare earth metal from a mixture of one or more rare earth metals, said method comprising:
preparing an ionic liquid using a process as defined herein; and
contacting an acidic solution of the rare earth metal with a composition which comprises the ionic liquid to form an aqueous phase and a non-aqueous phase into which the rare earth metal has been selectively extracted.

Typically, when rare earth metals are extracted from sources such as ores or waste materials, the resulting product is a mixture of rare earth metals dissolved in an aqueous acidic solution. In the method according to the present invention, rare earth metals may be selectively extracted directly from an aqueous acidic feed, negating the need to apply significant processing to the feed prior to extraction.

It will be appreciated that in order to form an aqueous phase and a non-aqueous phase when contacted with the acidic solution, the composition comprising an ionic liquid will be sufficiently hydrophobic such that a phase separation will occur between the aqueous solution and the composition.

By the use of the composition comprising an ionic prepared using a process as defined herein, it has been surprisingly found that increased selectivity and extractability may be obtained in the extraction of rare earth metals from an acidic solution. The combination of high extractability (indicated by distribution ratio) and selectivity (indicated by separation factors) is key to a commercially effective separation process because the number of separation stages necessary to produce a product may be reduced without sacrificing purity. For example, according to the method of the present invention, mixtures of dysprosium and neodymium may be separated with a selectivity (separation factor) of over 1000:1 in a single contact. This represents a substantial increase over known systems as reported in Table 1.

Without wishing to be bound by any particular theory, it is believed that the presence of the central nitrogen donor atom in the ionic liquid allows for differing binding strengths to different rare earth metals as a result of differing ionic radii due to lanthanide contraction. In this way, some rare earth metals are preferentially bound by the hydrophobic ionic liquid extractant, which results in effective intra-group separation of the rare earth metals. It is believed that the arrangement of this variable nitrogen binding as part of an ionic liquid provides the particularly effective extraction of rare earth metals described herein. Nonetheless, it will be appreciated that the ionic liquid comprising a nitrogen donor, whilst discriminating between different rare earth metals, must have additional electron donating groups appended in order to provide sufficient extractability.

It will be understood that the arrangement of the EDGs and the linkers L₂ in the ionic liquid is such that the EDGs and the central nitrogen atom are able to coordinate to a rare earth metal simultaneously. Preferably, when the nitrogen linking L₁ to each L₂ and one of the EDG both coordinate to a metal, the ring formed by the nitrogen, L₂, the EDG and the metal is a 5 or 6 membered ring, preferably a 5 membered ring.

The method for extracting a rare earth metal preferably further comprises recovering the rare earth metal from the non-aqueous phase. This recovery may be performed using any suitable means, however it is preferred that the rare earth metal is recovered from the non-aqueous phase by stripping with an acidic stripping solution.

It will be appreciated that the acidic stripping solution may be any acidic solution which liberates the rare earth metal from the ionic liquid. In most embodiments, the acidic stripping solution will be an aqueous acidic stripping solution and the acid will substantially remain in the aqueous phase on contact with the ionic liquid. Preferably, the acidic stripping solution comprises an aqueous hydrochloric acid or nitric acid solution.

The stripping of the rare earth metal may be conducted in any suitable manner. Preferably, the ionic liquid is contacted with an acidic stripping solution for 2 or more stripping cycles to completely strip the rare earth metal, more preferably 2 or 3 stripping cycles are used. In some embodiments, a single stripping cycle may be used. A “stripping cycle” as referred to herein will typically comprise contacting the acidic stripping solution with the composition, equilibrating for an amount of time, for example 5 to 30 minutes, and separating the aqueous and organic phases. A second cycle may be conducted by contacting the composition with another acidic stripping solution substantially free of rare earth metals.

One advantage of the ionic liquid extractant as described herein is that the rare earth metal may be stripped from the ionic liquid at a relatively high pH. This saves costs associated with both the amount and the strength of acid needed to strip the rare earth metals from the ionic liquid and the equipment necessary to handle such strong acids. In addition, it is possible to completely strip rare earth metals from the ionic liquid at a relatively high pH, whilst for many known extractants such as P507 it is difficult to completely strip heavy rare earth metals (e.g. Tm(III), Yb(III), Lu(III)) even at low pH.

Thus, the acidic stripping solution preferably has a pH of 0 or higher. In preferred embodiments, the acidic stripping solution has a pH of 1 or lower.

In preferred embodiments, the method comprises extracting a rare earth metal from a mixture of two or more rare earth metals. Preferably, the acidic solution comprises a first and a second rare earth metal, and the method comprises:

(a) preferentially partitioning the first rare earth metal into the non-aqueous phase.

Preferably, the method further comprises, in step (a), separating the non-aqueous phase from the acidic solution; and
(b) contacting the acidic solution depleted of the first rare earth metal with the composition which comprises an ionic liquid, and optionally recovering the second rare earth metal therefrom.

In some preferred embodiments the first rare earth metal is recovered from the non-aqueous phase in step (a), and said non-aqueous phase is recycled and used as the composition in step (b).

It will be appreciated that, because the extractability (distribution factor) for a particular rare earth metal varies with pH, it may be preferred to extract different rare earth metals at different pH levels. For example, the acidic solution may have a lower pH in step (a) in comparison to that in step (b). Preferably, the acidic solution has a pH of less than 3.5 in step (a), and the acidic solution has a pH of greater than 3.5 in step (b). Typically, 2 or 3 extraction cycles will be performed at a particular pH. Although the above embodiment describes extraction in only two different pH values, it will be appreciated that a separation of rare earth metals will usually be conducted across a range of pH values, with a gradual increase in pH and multiple extraction steps. For example, where three or more rare earth metals are separated, several separation steps may be conducted in across a particular pH range, for example from pH 1 to 4.

The acidic solution from which the rare earth metal is extracted may have any suitable pH. Preferably, the rare earth metal is extracted at a pH of more than 1, more preferably at a pH of from 2 to 4.

The pH level of the acidic solution of the rare earth metal may be adjusted in any suitable way, as is well known to those skilled in the art. For example, the pH level of the acidic solution may be altered by the addition of acid scavengers such as mildly alkaline solutions including sodium carbonate, sodium bicarbonate, ammonia, CO₂, amines or alcohols.

The above embodiments refer to the separation of a particular rare earth metal from another directly from the acidic solution of the rare earth metal at varying pH levels. However, it will be understood that any suitable extraction sequence may be used to separate rare earth metals. For example, two or more rare earth metals may be extracted from the acidic solution to the non-aqueous phase simultaneously at a higher pH, followed by back-extraction of the non-aqueous phase with acidic solutions having a lower pH to separate individual rare earth metals. Thus, all or only some of the rare earth metals present in the acidic solution may initially be extracted from the acidic solution using the composition comprising the ionic liquid.

It will be appreciated that the separation of certain pairs of rare earth metals are of particular importance due to their simultaneous recovery from valuable waste materials. For example, Nd and Dy are widely used in permanent magnets for numerous applications such as hard disks, MRI scanners, electric motors and generators. La and Eu are also an important pair due to their common use in lamp phosphors, other phosphors include Y and Eu (YOX phosphors); La, Ce and Tb (LAP phosphors); Gd, Ce and Tb (CBT phosphors); and Ce, Tb (CAT phosphors).

Thus, in preferred embodiments, the first rare earth metal is dysprosium, and the second rare earth metal is neodymium. In other preferred embodiments, the first rare earth metal is europium, and the second rare earth metal is lanthanum. In yet other preferred embodiments, the first rare earth metal is terbium, and the second rare earth metal is cerium.

The composition may be contacted with the acidic solution in any suitable manner and in any suitable ratio such that exchange of rare earth metals is achieved between the aqueous and non-aqueous phases.

The composition is preferably added to the acidic solution in a volume ratio of from 0.5:1 to 2:1, preferably 0.7:1 to 1.5:1, more preferably 0.8:1 to 1.2:1, for example 1:1. Nonetheless, it will be appreciated that the volume ratio will vary depending on the manner in which the acidic solution is contacted with the composition comprising the ionic liquid.

Preferably, prior to contacting the composition with the acidic solution of the rare earth metal the composition is equilibrated with an acidic solution having the same pH as the acidic solution of the rare earth metal. In this way, the mixture of the composition and the acidic solution will generally remain at the desired pH level during the extraction.

The composition may be contacted with the acidic solution of the rare earth metal under any conditions suitable for extracting the rare earth metal.

It will be appreciated that the temperature employed during contacting of the acidic solution with the composition comprising the ionic liquid may be any suitable temperature and may vary according to the viscosity of the composition comprising the ionic liquid. For example, where a higher viscosity composition is used, a higher temperature may be necessary in order to obtain optimal results.

Preferably, the acidic solution is contacted with the composition at ambient temperature, i.e. without external heating or cooling. It will nonetheless be appreciated that temperature changes may naturally occur during the extraction as a result of contacting the composition with the acidic solution.

The composition may be contacted with the acidic solution of the rare earth metal for any length of time suitable to facilitate extraction of the rare earth metal into the non-aqueous phase. Preferably, the length of time will be such that an equilibrium is reached and the proportions of rare earth metal in the aqueous and non-aqueous phases are constant. In preferred embodiments, the method comprises contacting the acidic solution of the rare earth metal and the composition for from 1 to 40 minutes, preferably from 5 to 30 minutes.

Preferably, the method comprises contacting and physically mixing the acidic solution of the rare earth metal and the composition. Such mixing will usually speed up extraction of the rare earth metal. Any suitable apparatus may be used to achieve this and mixing apparatus is well known in the art. For example, the mixture may be mixed using an agitator or stirrer. The mixing apparatus may comprise equipment specifically designed for multi-phase mixing such as high shear devices. Alternatively, mixing may comprise shaking the mixture, for example, using a wrist action shaker.

The separation of the aqueous and non-aqueous phases may be performed by any suitable method, for example by use of small scale apparatus such as a separating funnel or Craig apparatus. It will be appreciated that the phases will normally be allowed to settle prior to separation. Settling may be under gravity or preferably accelerated by the use of additional equipment such as centrifuge. Alternatively, aqueous and non-aqueous phases may be separated by the use of equipment which both contacts and separates the phases, for example a centrifugal extractor, a pulsed column, or a combined mixer-settler.

It will be understood that in order to extract or separate some rare earth metals, multiple extractions and separations may be performed. This may involve multiple extractions of the acidic solution of the rare earth metal with the composition or multiple back-extractions of the non-aqueous phase with an aqueous acidic solution. In accordance with the present invention, fewer steps are required to separate rare earth metals due to the ionic liquid extractant giving separation factors and distribution ratios above those typically found in previous systems.

It will be understood that the composition may comprise the ionic liquid as defined herein in combination with a diluent. Typically, a diluent may be used in order to decrease the viscosity of the composition where the ionic liquid has a high viscosity, which limits its practical use in liquid-liquid extraction. A diluent may also be used to save costs where the diluent is cheaper to produce than the ionic liquid. It will be understood that any diluent added to the composition will be sufficiently hydrophobic so as to allow the separation of the composition and the acidic solution of the rare earth metal into an aqueous and non-aqueous phase. In some embodiments, the diluent may enhance the hydrophobicity of the composition.

Thus, in preferred embodiments, the composition further comprises a lower viscosity ionic liquid. The term “lower viscosity ionic liquid” will be understood to mean that this ionic liquid has a lower viscosity than the ionic liquid extractant described previously. As mentioned, it will be understood that the lower viscosity ionic liquid will be sufficiently hydrophobic so as to allow the separation of the composition and the acidic solution of the rare earth metal into an aqueous and non-aqueous phase. It will also be appreciated that the hydrophobicity may be provided by either of the cation or anion of the lower viscosity ionic liquid, or by both.

By the use of an ionic liquid as a diluent, the decreased volatility and flammability offered by the ionic liquid extractant may be maintained to give a potentially safer and more environmentally friendly rare earth metal extraction process.

In preferred embodiments, the cation of the lower viscosity ionic liquid is selected from ammonium, benzimidazolium, benzofuranium, benzothiophenium, benzotriazolium, borolium, cinnolinium, diazabicyclodecenium, diazabicyclononenium, 1,4-diazabicyclo[2.2.2]octanium, diazabicyclo-undecenium, dithiazolium, furanium, guanidinium, imidazolium, indazolium, indolinium, indolium, morpholinium, oxaborolium, oxaphospholium, oxazinium, oxazolium, iso-oxazolium, oxothiazolium, phospholium, phosphonium, phthalazinium, piperazinium, piperidinium, pyranium, pyrazinium, pyrazolium, pyridazinium, pyridinium, pyrimidinium, pyrrolidinium, pyrrolium, quinazolinium, quinolinium, iso-quinolinium, quinoxalinium, quinuclidinium, selenazolium, sulfonium, tetrazolium, thiadiazolium, iso-thiadiazolium, thiazinium, thiazolium, iso-thiazolium, thiophenium, thiuronium, triazinium, triazolium, iso-triazolium and uronium groups.

Preferably the cation of the lower viscosity ionic liquid is selected from phosphonium, imidazolium and ammonium groups.

In some preferred embodiments, the cation of the lower viscosity ionic liquid is selected from.

[N(R³)(R⁴)(R⁵)(R⁶)]⁺ and [P(R³)(R⁴)(R⁵)(R⁶)]⁺,
wherein: R³, R⁴, R⁵ and R⁶ are each independently selected from optionally substituted C₁₋₂₀ alkyl, C₃₋₈ cycloalkyl and C₆₋₁₀ aryl groups.

In more preferred embodiments, the cation of the lower viscosity ionic liquid is [P(R³)(R⁴)(R⁵)(R⁶)]⁺, wherein R³, R⁴, R⁵ are selected from C₁₋₁₀ alkyl, preferably C₂₋₆ alkyl, and R⁶ is selected from C₄₋₂₀ alkyl, preferably C₈₋₁₄ alkyl. For example, the cation of the lower viscosity ionic liquid may be selected from triethyloctyl phosphonium (P₂₂₂₍₈₎]⁺), tributyloctyl phosphonium (P₄₄₄₍₈₎]⁺), trihexyloctyl phosphonium (P₆₆₆₍₈₎]⁺), trihexyldecyl phosphonium (P₆₆₆₍₁₀₎]⁺), and trihexyltetradecyl phosphonium (P₆₆₆₍₁₄₎]⁺).

In other more preferred embodiments, the cation of the lower viscosity ionic liquid is

[N(R³)(R⁴)(R⁵)(R⁶)]⁺, wherein R³, R⁴, R⁵ are selected from C₄₋₁₄ alkyl, preferably C₆₋₁₀ alkyl, and R⁶ is selected from C₁₋₄ alkyl, preferably C₁₋₂ alkyl. For example, the cation of the lower viscosity ionic liquid may be selected from trioctylmethyl ammonium, tris(2-ethylhexyl) methyl ammonium, and tetrabutyl ammonium.

In other preferred embodiments, the cation of the lower viscosity ionic liquid is selected from imidazolium cations substituted with one or more C₁₋₂₀ alkyl, C₃₋₈ cycloalkyl and C₆₋₁₀ aryl groups, preferably substituted with two C₁₋₁₀ alkyl groups, more preferably substituted with one methyl group and one C₁₋₁₀ alkyl group. For example, the cation of the lower viscosity ionic liquid may be selected from 1-butyl-3-methyl imidazolium, 1-hexyl-3-methyl imidazolium and 1-octyl-3-methyl imidazolium.

It will be understood that any suitable anionic group may be used as the anion of the lower viscosity ionic liquid. Preferably, the anion of the lower viscosity ionic liquid is as described previously in relation to the anionic group [X⁻]. For example, it is most preferred that the anion of the lower viscosity ionic liquid is a non-coordinating anion as described previously. It will be appreciated that there may be an excess of anions from the lower viscosity ionic liquid in comparison to the ionic liquid extractant. Therefore, it is especially preferred that the anion of the lower viscosity ionic liquid is a non-coordinating anion.

For this reason, it is preferable to limit the total amount of halide or pseudohalide anions in the composition. For example, in preferred embodiments the composition comprises less than 25% halide or pseudohalide anions as a proportion of the total anions, preferably less than 20%, more preferably less than 15%, most preferably less than 10%, for example less than 5%. In some embodiments, the composition is substantially free of halide or pseudohalide anions.

The composition may alternatively or additionally further comprise one or more non-ionic liquid diluents. For example, in some preferred embodiments, the composition further comprises one or more organic solvents. It will be understood that suitable organic solvents will include hydrophobic and non-coordinating solvents. The term “non-coordinating solvent” used herein, which is common in the field of metal coordination chemistry, is intended to mean a solvent that does not coordinate with metal atoms or ions, or does so only weakly.

Suitable organic solvents include but are not limited to hydrocarbon solvents such as C₁₋₂₀ alkanes, alkenes or cycloalkanes, aromatic solvents such as toluene or benzene, C₆₊ alcohols such as n-hexanol, etheric solvents such as diethyl ether, dipropyl ether, dibutyl ether and methyl-t-butyl ether, or halogenated solvents such as tetrachloromethane, tetrachloroethane, chloroform, dichloromethane, chlorobenzene, or fluorobenzene. Preferably the organic solvent is a hydrocarbon solvent.

The ionic liquid may be present in the composition in any concentration suitable for extracting rare earth metals and it will be appreciated that this concentration will vary depending on the particular application and pH. In particular, it will be appreciated that for the separation of rare earth metals a competitive separation is desirable. For example the concentration of the ionic liquid should be low enough to avoid the extraction of all rare earth metals present. Therefore, the concentration of the ionic liquid will typically depend on the concentration of rare earth metals to be extracted and the pH at which the separation is conducted. In some preferred embodiments, the ionic liquid is present in the composition in a concentration of at least 0.001 M, preferably from 0.005 M to 0.01 M.

In other embodiments, the composition may consist essentially of the ionic liquid.

It will be appreciated that the concentration of the ionic liquid in the composition may be varied in order to achieve a particular target viscosity for the composition. It will also be appreciated that the character of the lower viscosity ionic liquid or other diluent may be varied in order to obtain a particular viscosity level.

In preferred embodiments, the viscosity of the composition is in the range of from 50 to 500 mPa·s at 298 K, when the composition comprises a solution of the ionic liquid in a lower viscosity ionic liquid. When the ionic liquid is in a solution of an organic solvent, it will be appreciated that the composition will likely have a lower viscosity, for example, less than 50 mPa·s. Viscosity may be measured by any suitable method, for example viscosity may be measured using a rotating disk viscometer with variable temperature.

In some embodiments, the acidic solution is obtainable by leaching the rare earth metal from its source using an acid, for example a mineral acid such as hydrochloric, nitric, perchloric or sulfuric acid, typically hydrochloric or nitric acid. Preferably, the source of the rare earth metal is a mineral or a waste material. However, it will be appreciated that the acidic solution of the rare earth metal or mixture of rare earth metals may be obtained in any suitable way from any rare earth metal source.

The concentration of rare earth metals in the acidic solution is typically from 60 to 2000 ppm. Nonetheless, it will be appreciated that any suitable concentration of rare earth metals in the acid solution may be used.

Typically, rare earth metals are obtained from rare earth ores, which are mined and processed by a variety of methods depending on the particular ore. Such processes are well known in the art. Usually, following mining such processes may include steps such as grinding, roasting to remove carbonates, chemical processing (e.g alkali/hydroxide treatment), and ultimately leaching with acid to obtain an aqueous acidic solution containing a mixture of rare earth metals.

Examples of rare earth metal bearing minerals contained in rare earth ores are aeschynite, allanite, apatite, bastnasite, brannerite, britholite, eudialyte, euxenite, fergusonite, gadolinite, kainosite, loparite, monazite, parisite, perovskite, pyrochlore, xenotime, yttrocerite, huanghoite, cebaite, florencite, synchysite, samarskite, and knopite.

Rare earth metals may also increasingly be obtained from recycled materials. As global demand for rare earth metals grows, it is increasingly attractive to obtain earth metals from recycled waste materials, particularly in countries with a lack of minable rare earth ore deposits. Rare earth waste materials may be obtained from various sources, for example direct recycling of rare earth scrap/residues from pre-consumer manufacturing, “urban mining” of rare earth containing end of life products, or landfill mining of urban and industrial waste containing rare earths. As rare earth metals are increasingly being used in consumer products, the amount of rare earth metals that can be obtained from such waste materials is also growing.

Waste materials that may contain rare earth metals include, magnetic swarf and rejected magnets, rare earth containing residues from metal production/recycling (e.g. postsmelter and electric arc furnace residues or industrial residues such as phosphogypsum and red mud), phosphors such as those in fluorescent lamps, LEDs, LCD backlights, plasma screens and cathode ray tubes, permanent magnets (e.g. NdFeB) such as those used in automobiles, mobile phones, hard disk drives, computers and peripherals, electronic kitchen utensils, hand held tools, electric shavers, industrial electric motors, electric bicycles, electric vehicle and hybrid vehicle motors, wind turbine generators, nickel-metal hydride batteries such as are used for rechargeable batteries and electric and hybrid vehicle batteries, glass polishing powders, fluid cracking catalysts and optical glass. Major end-of-life waste material sources of rare earths in terms of value are permanent magnets, nickel-metal hydride batteries and lamp phosphors, as well as scrap in the form of magnetic swarf waste.

Rare earth metals will usually be extracted from waste materials by leaching with mineral acids and optionally further processing to remove impurities such as transition metals. This results in an acidic solution of the rare earth metals, which may be used as a source for separation and purification of the individual rare earth metals.

Thus, it is an advantage of the present invention that rare earth metals may be extracted with high selectivity and extractability directly from an acidic solution of the rare earth metal, which may be conveniently obtained from the extraction process of an ore or a waste material.

A further aspect provides a process for preparing a cationic species [Cat⁺] as defined previously, said process comprising carrying out the following reaction:

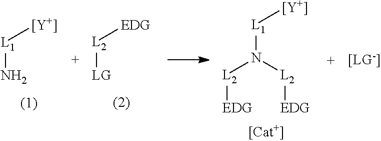

where: LG represents a leaving group;

wherein the process is carried out in a sealed reactor at a temperature of greater than 100° C.

It will be appreciated that the process, including the process steps and conditions, and the nature of L₁, L₂, LG, [Y⁺] and EDG may be substantially as described previously herein in relation to the previous aspect.

According to this aspect [Y⁺] is preferably phosphonium or ammonium, preferably phosphonium.

The process may comprise the step of forming reagent (1) according to the following reaction:

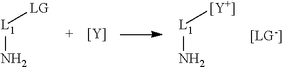

where [LG⁻] may optionally be exchanged with a different anion [X⁻] before reacting further with reagent (2).

Reagent (2) may preferably be used in an amount of from 1 to 6 molar equivalents, preferably from 1 to 3 molar equivalents and more preferably from 1.5 to 2.5 molar equivalents as compared to reagent (1).

The present invention will now be illustrated by way of the following examples and with reference to the following figures in which:

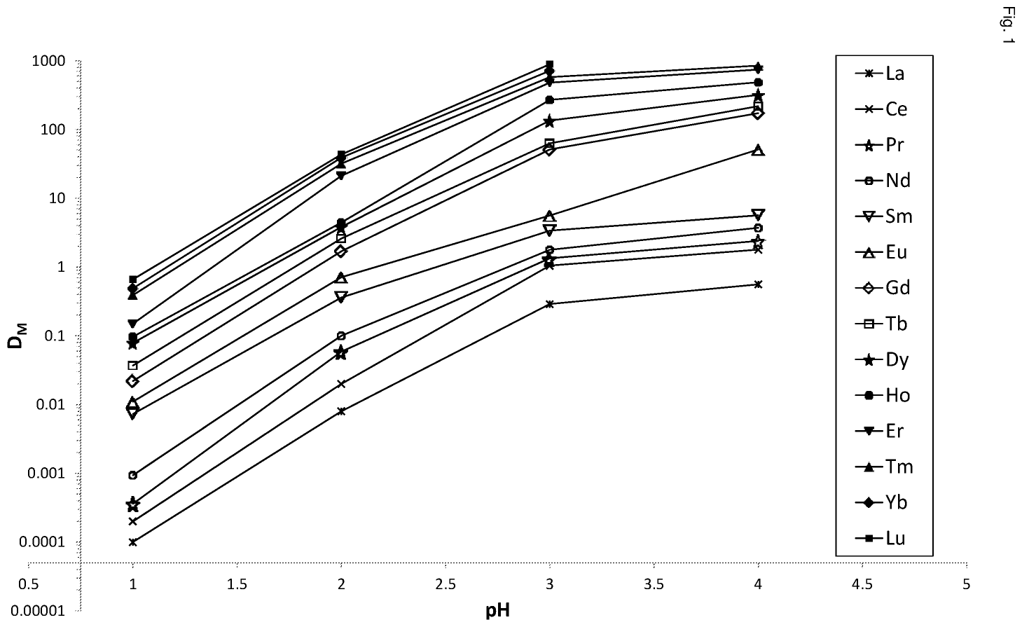

FIG. 1 is a graph showing the distribution factors for the extraction of a selection of rare earth metals according to an embodiment of the present invention; and

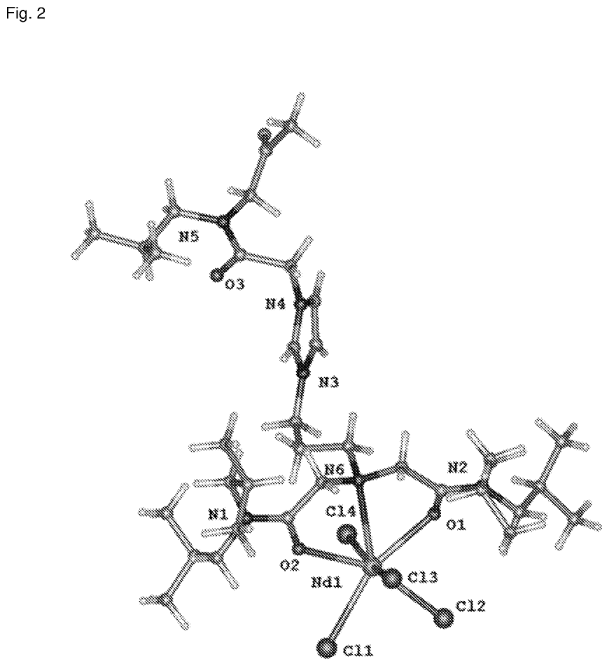

FIG. 2 shows the crystal structure of the [MAIL]⁺ cation coordinating to Nd after extraction from an acidic (HCl) solution containing NdCl₃.6H₂O.

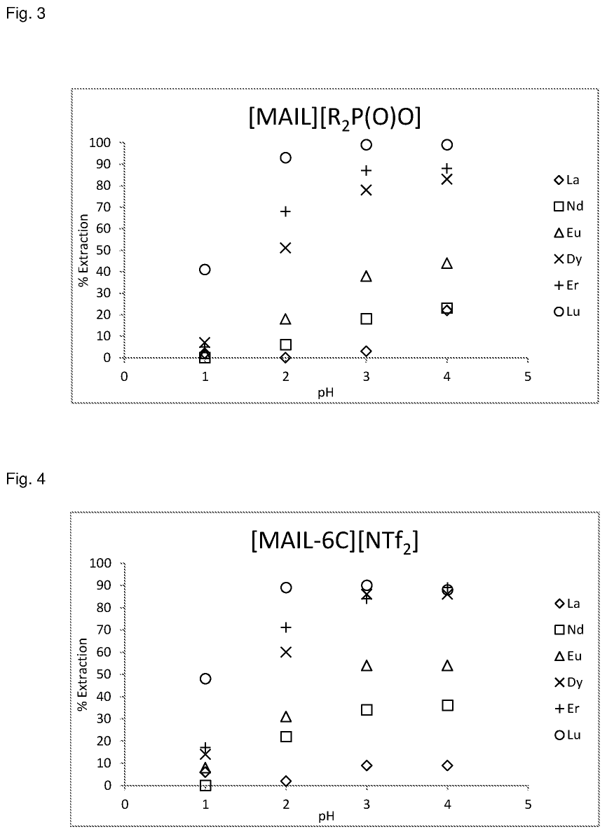

FIG. 3 is a graph showing extraction of a selection of rare earth metals using [MAIL⁺][R₂P(O)O⁻].

FIG. 4 is a graph showing extraction of a selection of rare earth metals using [MAIL-6C⁺][NTf₂⁻].

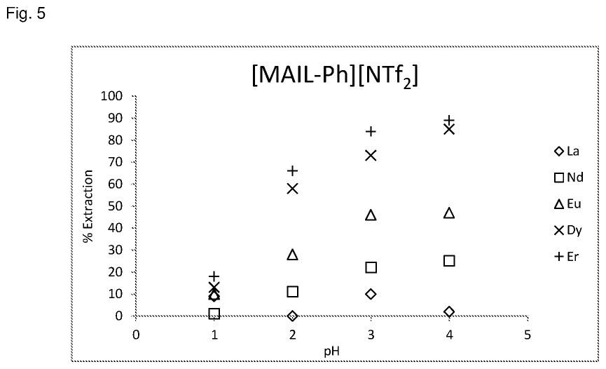

FIG. 5 is a graph showing extraction of a selection of rare earth metals using [MAIL-Ph⁺][NTf₂⁻].

### EXAMPLES

### Example 1: Synthesis of Ionic Liquid

General Procedure for the Synthesis of an Ionic Liquid According to Embodiments of the Invention

A reaction mixture comprising reagent (1) (e.g. a 1-(amino alkyl)-imidazole), reagent (2) (e.g. an N,N-dialkyl-2-haloacetamide, e.g. in a molar ratio of 3:1 with reagent (1)) and a base (e.g. trimethylamine, e.g. in a molar ratio of 4:1 with regent (1)) is mixed in a solvent (e.g.

trichloromethane) at a temperature of greater than 100° C. (e.g. 125 to 160° C.).

After cooling, the organic phase is washed with acid (e.g. HCl) then base (e.g. Na₂CO₃) and finally with water (e.g. deionised water) until the aqueous phase showed a neutral pH. Solids will typically not be present in the organic phase, so filtering is generally not required. The solvent is removed (e.g. under high vacuum) from the purified ionic liquid to give the ionic liquid product in isolated form.

This ionic liquid may be used as it is, otherwise the anion is exchanged with a different anion (e.g. bistriflimide) by reacting the desired anion (e.g. in the form of an alkali metal salt) with the ionic liquid in a solvent (e.g. trichloromethane).

Synthesis of an Imidazolium Ionic Liquid

[MAIL⁺][NTf₂⁻]:

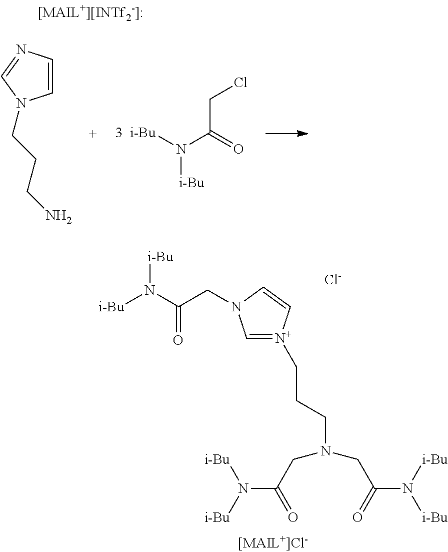

1-(3-Aminopropyl)-imidazole (0.05 mol), N,N-diisobutyl-2-chloroacetamide (0.15 mol), triethylamine (0.20 moles) and chloroform were added to a glass pressure tube. The tube was sealed, and the reaction was stirred for 4 hours at 130° C. The reaction mixture was then cooled and, without filtering, successively washed with 0.1 M HCl, 0.1 M Na₂CO₃ and deionized water. The solvent was removed from the neutralised organic phase at 8 mbar (6 mm Hg) and finally at 60° C. and 0.067 mbar (0.05 mmHg). The ionic liquid [MAIL⁺]Cl⁻ was recovered as a highly viscous yellow liquid.

Ionic liquid [MAIL⁺]Cl⁻ (0.025 mol) was dissolved in chloroform and lithium bis-(trifluoromethane) sulfonamide (LiNTf₂) (0.03 mol) dissolved in water was added. The reaction mixture was stirred for 1 hour and then the organic phase was repeatedly washed with deionized water. Finally the solvent was removed from the organic phase under vacuum (0.13 mbar ,0.1 mm Hg) at 65° C. to yield the bistriflimide anion form of the ionic liquid ([MAIL⁺][NTf₂⁻]).

The synthesis was repeated using the same method, but with modified temperatures and reaction times. The yield obtained in each experiment is provided in the table below:

| | Temperature | Time | Yield |
|---|---|---|---|
| Experiment | (° C.) | (hours) | (%) |
| 1 | 120 | 1 | 60 |
| 2 | 120 | 4 | 66 |
| 3 | 130 | 1 | 73 |
| 4 | 130 | 4 | 79 |
| 5 | 140 | 1 | 74 |
| 6 | 140 | 4 | 78 |

It can be seen that a high yield is obtained at the temperatures tested, with particularly high yields obtained at temperatures over 125° C. The reaction also proceeded extremely quickly, with similar yields obtained over a period of 1 and 4 hours. [MAI L-6C⁺][NTf₂⁻]:

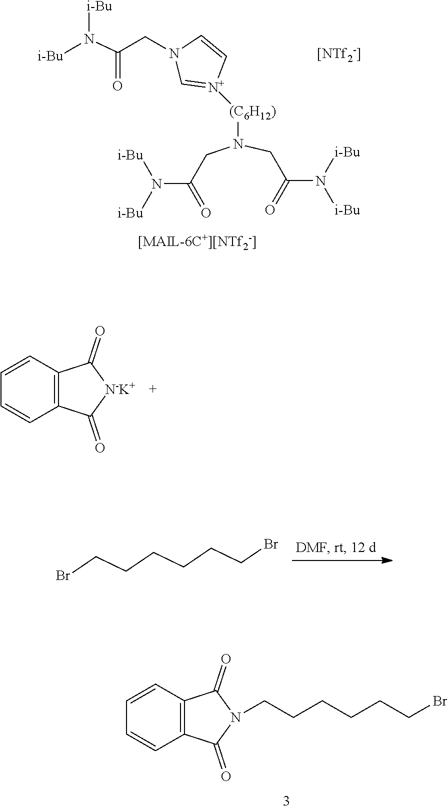

A mixture of potassium pthalimide (10.0 g, 54.0 mmol) and 1,6-dibromobutane (9.97 mL, 64.8 mmol) in dry DMF (100 mL) was stirred at room temperature for 12 days. The mixture was concentrated and extracted with chloroform (3×30 mL) and washed with deionised water (3×80 mL) and brine (100 mL). The organic layer was dried over magnesium sulfate and concentrated to give a white syrup. The syrup was triturated with hexanes, filtered and dried to give a white solid product (3) (14.3 g, 85%).

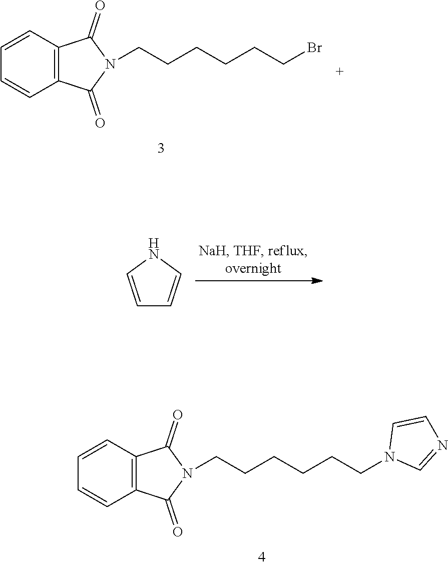 [SMILES: O=C1c2ccccc2C(=O)N1CCCCCCBr ; c1cc[nH]c1 ; O=C1c2ccccc2C(=O)N1CCCCCCn1ccnc1]

To NaH (0.645 g, 26.9 mmol) in THF was added at 0° C. under N_{2,} imidazole (1.21 g, 17.7 mmol) in THF was added over 30mins, and stirred for a further 30mins at 0° C. 3 (5.00 g. 16.1 mmol) in THF was added at 0° C. and the mixture stirred for 1 hour at room temperature, then refluxed at 70° C. overnight. The mixture was filtered and the residual NaBr was washed with THF. The filtrate was concentrated to give a syrup which was dissolved in DCM to give a yellow solution which was then washed with water and dried over sodium sulfate and triturated with hexanes to precipitate a white solid which was filtered and washed with hexanes (4) (1.52 g, 32%).

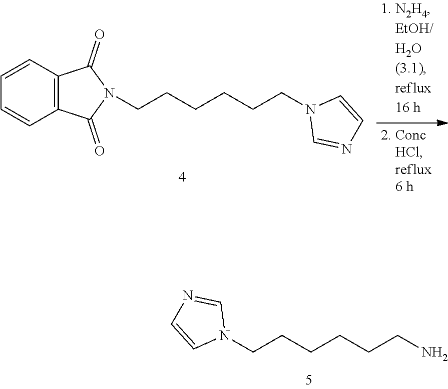 [SMILES: O=C1c2ccccc2C(=O)N1CCCCCCn1ccnc1 ; NCCCCCCn1ccnc1]

4 (0.750 g, 2.54 mmol) was dissolved in a EtOH:H₂O mixture (160 mL, 3:1) and hydrazine hydrate (50-60%, 0.174 mL, 5.55 mmol) was added at room temperature and the mixture refluxed overnight. The solution was cooled to room temperature and concentrated HCl (2 mL) was added, the reaction mixture changed from colourless to yellow to red to light yellow during the addition. The mixture was stirred at reflux for 6 hours and filtered. The solution was concentrated and dissolved in distilled water to give a yellow solution. Sodium hydroxide was added until the mixture reached pH 11, it was then extracted with chloroform (4×40 mL), dried over magnesium sulfate and concentrated to give an orange oil (5) (0.329 g, 78%).

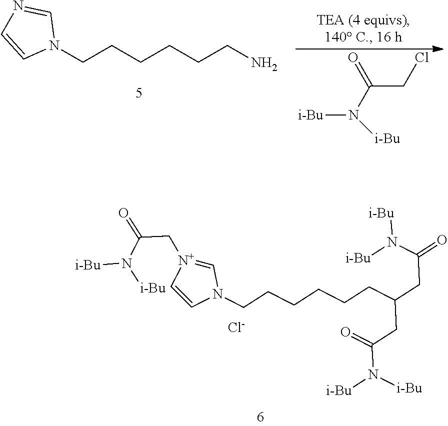

To a high pressure vessel was added 5 (0.257 g, 1.54 mmol), triethylamine (0.623 g, 6.16 mmol), N,N-diisobutyl-2-chloroacetamide (0.950 g, 4.62 mmol) and chloroform (5 mL). The vessel was stoppered and stirred at 140° C. on an oil bath for 16 hours. The reaction mixture was washed with pH 1 HCl (40 mL), Na₂CO₃ (2×40 mL) then water (4×40 mL). The organic layer was dried over magnesium sulfate and concentrated in vacuo to give a viscous dark brown liquid (6) (0.648 g, 59%).

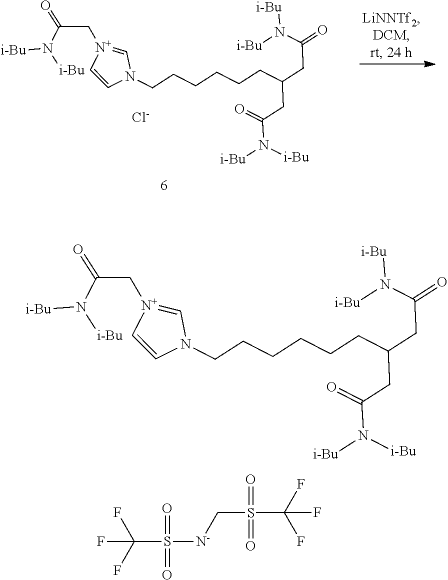

To a round bottom flask was added 6 (0.6255 g, 0.88 mmol) followed by DCM (50 mL). LiNTf₂ (0.7572 g, 2.64 mmol) was added followed by water (50 mL). The reaction mixture was stirred at room temperature for 24 hours. The aqueous layer was removed and the organic layer washed with deionised water (4×40 mL). The organic layer was dried over magnesium sulfate and concentrated. The product was dried overnight to give a black viscous liquid, [MAIL-6C⁺][NTf₂⁻], (0.7467 g, 89%).

[MAIL-Ph⁺][NTf₂⁻]:

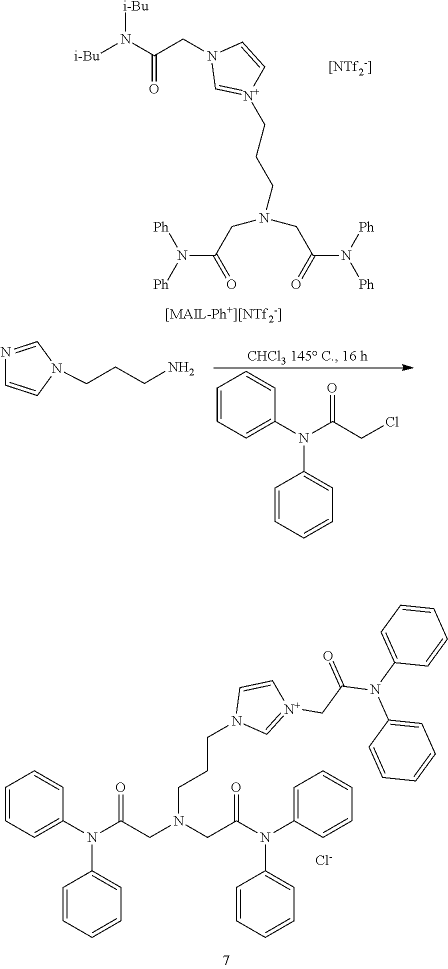

To a high pressure vessel was added 1-(3-aminopropyl)imidazole (0.200 g, 1.60 mmol), triethylamine (0.647 g, 6.39 mmol), 2-chloro-N,N-diphenylacetamide (1.18 g, 4.49 mmol) and chloroform (5 mL). The vessel was stoppered and stirred at 145° C. on an oil bath for 16 hours. The reaction mixture was washed with pH 1 HCl (15 mL), then water (4×150 mL).

The organic layer was dried over magnesium sulfate and concentrated in vacuo to give an orange/brown solid (7) (0.883 g, 70%).

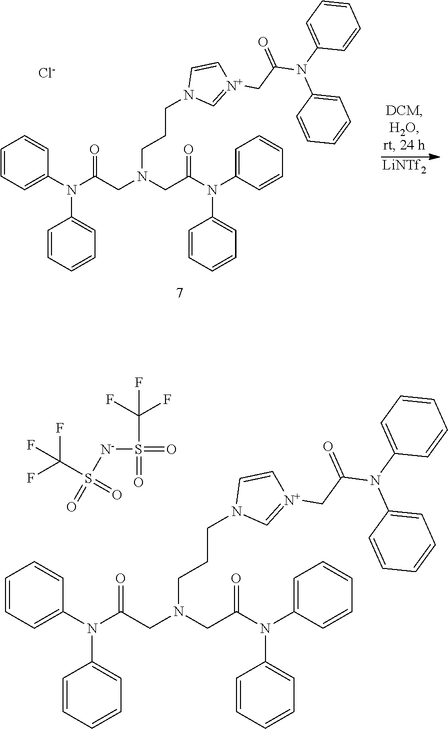 [SMILES: O=C(CN(CCCn1cc[n+](CC(=O)N(c2ccccc2)c2ccccc2)c1)CC(=O)N(c1ccccc1)c1ccccc1)N(c1ccccc1)c1ccccc1 ; [Cl-] ; O=C(CN(CCCn1cc[n+](CC(=O)N(c2ccccc2)c2ccccc2)c1)CC(=O)N(c1ccccc1)c1ccccc1)N(c1ccccc1)c1ccccc1 ; O=S(=O)([N-]S(=O)(=O)C(F)(F)F)C(F)(F)F ; "LiNTf2"]

To a 50 mL round-bottom flask was added 7 (0.444 g, 0.560 mmol) followed by DCM (20 mL). LiNTf₂ (0.484 g, 1.69 mmol) was added followed by deionised water (20 mL). The reaction mixture was stirred at room temperature for 24 hours. The aqueous layer was removed and the organic layer washed with deionised water (5×15 mL). The organic layer was dried over magnesium sulfate and concentrated. The product was dried overnight to give a viscous brown liquid, [MAIL-Ph⁺][NTf₂⁻], (0.351 g, 65%).

The phosphinate ionic liquid [MAIL⁺][R₂P(O)O⁻](R=2,4,4-trimethylpentyl) was also synthesised by ion exchange.

Comparative Synthesis of the Imidazolium Ionic Liquid 1-(3-Aminopropyl)-imidazole (0.05 mol) was added to of N,N-diisobutyl-2-chloroacetamide (0.15 mol) in a 500 ml three necked round bottom flask. Triethylamine (0.11 moles) was then added along with chloroform (200 ml). The reaction was stirred for 6 hours at room temperature and then stirred at 60 to 70° C. for 7 days. The reaction mixture was then cooled and after filtration it was successively washed with 0.1 M HCl, 0.1 M Na₂CO₃ and deionized water. The solvent was removed from the neutralised organic phase at 8 mbar (6 mm Hg) and finally at 60° C. and 0.067 mbar (0.05 mmHg). The ionic liquid [MAIL⁺]Cl⁻ was recovered as a highly viscous yellow liquid.

Ionic liquid [MAIL⁺]Cl⁻ (0.025 mol) was dissolved in chloroform and lithium bis-(trifluoromethane) sulfonamide (LiNTf₂) (0.03 mol) was added. The reaction mixture was stirred for 1 hour and then the organic phase was repeatedly washed with deionized water. Finally the solvent was removed from the organic phase under vacuum (0.13 mbar ,0.1 mm Hg) at 65° C. to yield the bistriflimide anion form of the ionic liquid ([MAIL⁺NTf₂⁻]).

Synthesis of Phosphonium Ionic Liquids

[MAIL-PPh₃⁺][NTf₂⁻]:

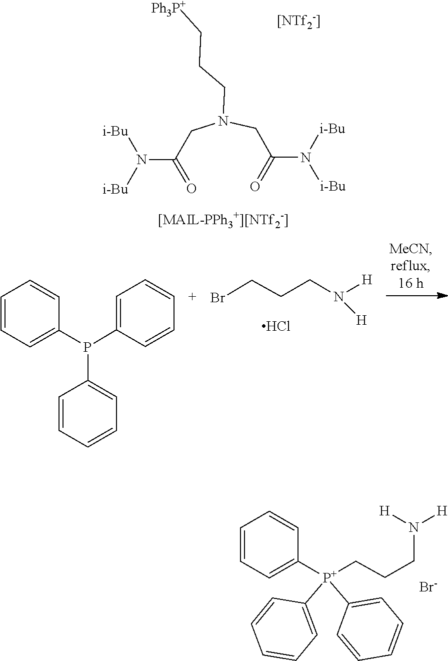

To a 50 mL round-bottom flask equipped with a magnetic stir bar triphenylphosphine (0.836 g, 3.19 mmol), 3-bromopropylamine hydrobromide (1.00 g, 4.57 mmol), and acetonitrile (25 mL) were added. The suspension was then heated and stirred at reflux for 16 hours. The reaction was cooled to room temperature, and the solvent was removed under reduced pressure, and the resulting white solid was then dried in vacuo, and used in subsequent steps without further purification (1.01 g, 79%).

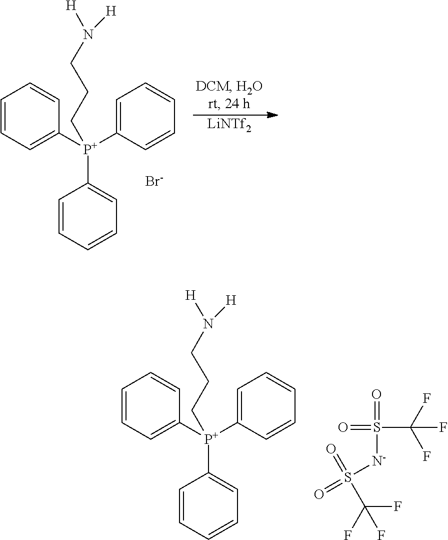

To a 50 mL round-bottom flask was added (3-Aminopropyl)(triphenyl)phosphonium bromide (1.01 g, 0.252 mmol) followed by DCM (20 mL). LiNTf₂ (2.17 g, 7.55 mmol) was added followed by deionised water 20 mL). The reaction mixture was stirred at room temperature for 24 hours. The aqueous layer was removed and the organic layer washed with deionised water (5×15 mL). The organic layer was dried over magnesium sulfate and concentrated. The product was dried overnight to give a white solid (1.26 g, 84%).

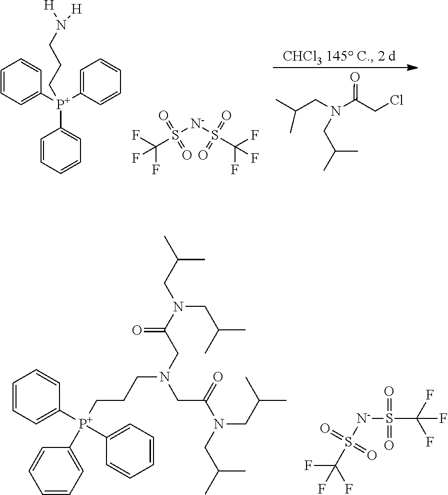 [SMILES: NCCC[P+](c1ccccc1)(c1ccccc1)c1ccccc1 ; O=S(=O)([N-]S(=O)(=O)C(F)(F)F)C(F)(F)F ; CC(C)CN(CC(C)C)C(=O)CCl ; CC(C)CN(CC(C)C)C(=O)CN(CCC[P+](c1ccccc1)(c1ccccc1)c1ccccc1)CC(=O)N(CC(C)C)CC(C)C ; O=S(=O)([N-]S(=O)(=O)C(F)(F)F)C(F)(F)F]

To a high pressure vessel was added (3-Aminopropyl)(triphenyl)phosphonium bistriflimide (0.200 g, 0.333 mmol), triethylamine (0.135 g, 1.33 mmol), N,N-diisobutyl-2-chloroacetamide (0.137 g, 0.666 mmol) and chloroform (5 mL). The vessel was stoppered and stirred at 145° C. on an oil bath for 48 hours. The reaction mixture was washed with pH 1 HCl (15 mL), then water (4×150 mL). The organic layer was dried over magnesium sulfate and concentrated in vacuo to give a viscous dark brown liquid, [MAIL-PPh₃⁺][NTf₂⁻], (0.282 g, 90%).

[MAIL-PPh₃⁺][R₂P(O)O⁻]:

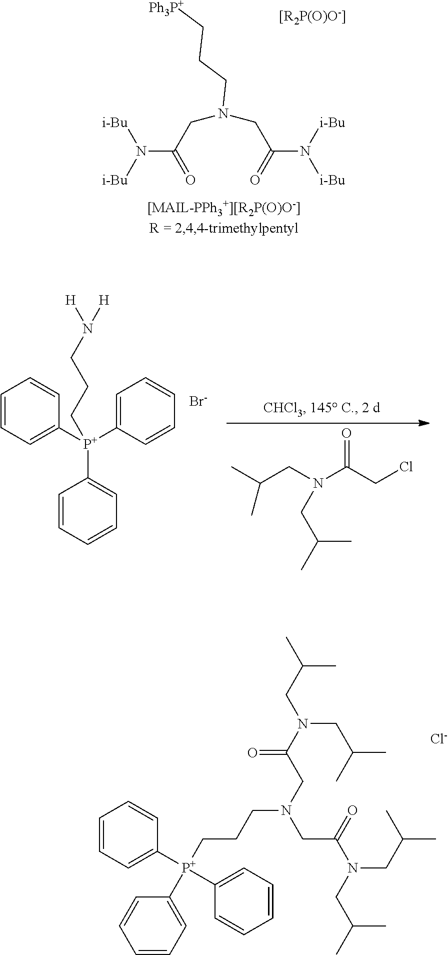

To a high pressure vessel was added (3-Aminopropyl)(triphenyl)phosphonium bromide (1.01 g, 2.53 mmol), triethylamine (1.03 g, 10.1 mmol), N,N-diisobutyl-2-chloroacetamide (1.04 g, 5.07 mmol) and chloroform (5 mL). The vessel was stoppered and stirred at 145° C. on an oil bath for 48 hours. The reaction mixture was washed with pH 1 HCl (15 mL), then water (4×150 mL). The organic layer was dried over magnesium sulfate and concentrated in vacuo to give a viscous dark brown liquid (0.981 g, 56%).

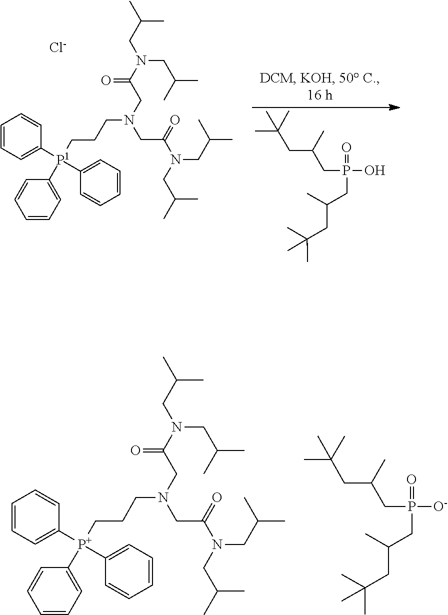

To a 50 mL round-bottom flask was added the phosphonium diamide (0.898 g, 1.29 mmol) followed by DCM (20 mL). R₂P(O)OH (R=2,4,4-trimethylpentyl) (0.356 g, 1.29 mmol) was added followed by a KOH solution (40%, 20 mL). The reaction mixture was stirred at 50° C. for 16 hours. The aqueous layer was removed and the organic layer washed with deionised water (5×15 mL). The organic layer was dried over magnesium sulfate and concentrated. The product was dried overnight to give a white solid, [MAIL-PPh₃⁺][R₂P(O)O⁻], (0.943 g, 77%). [MAIL-P₄₄₄⁺][NTf₂⁻]:

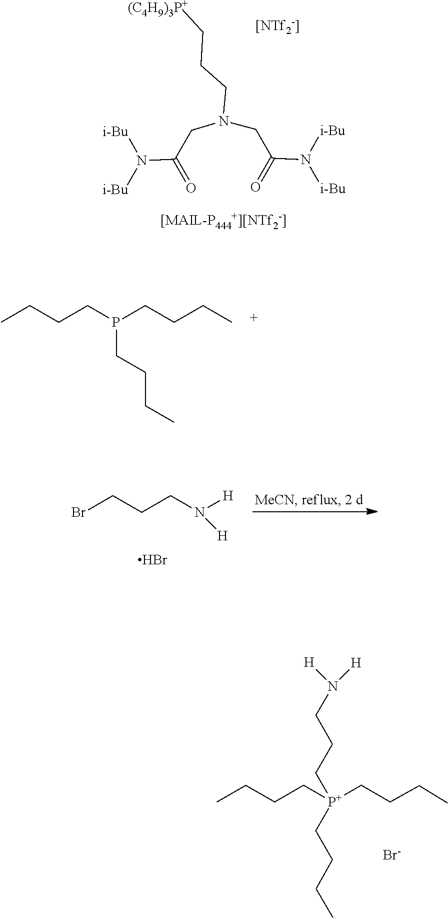

To a 50 mL round-bottom flask equipped with a magnetic stir bar tributylphosphine (0.823 g, 4.07 mmol), 3-bromopropylamine hydrobromide (0.890 g, 4.07 mmol), and acetonitrile (25 mL) were added. The suspension was then heated and stirred at reflux for 48 hours. The reaction was cooled to room temperature, and the solvent was removed under reduced pressure, and the resulting oil was then dried in vacuo, and used in subsequent steps without further purification (1.24 g, 89%).

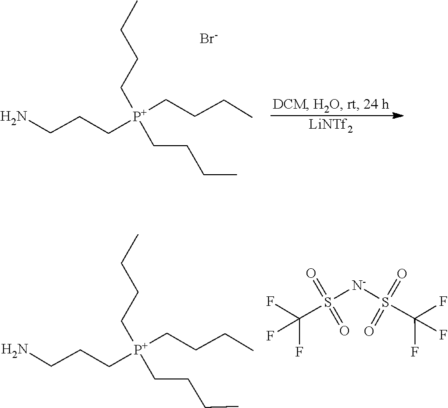 [SMILES: CCCC[P+](CCCC)(CCCC)CCCN ; [Br-] ; CCCC[P+](CCCC)(CCCC)CCCN ; O=S(=O)([N-]S(=O)(=O)C(F)(F)F)C(F)(F)F ; "LiNTf2"]

To a 50 mL round-bottom flask was added (3-Aminopropyl)(tributyl)phosphonium bromide (0.559 g, 1.64 mmol) followed by DCM (20 mL). LiNTf₂ (1.41 g, 4.93 mmol) was added followed by deionised water (20 mL). The reaction mixture was stirred at room temperature for 24 hours. The aqueous layer was removed and the organic layer washed with deionised water (5×15 mL). The organic layer was dried over magnesium sulfate and concentrated. The product was dried overnight to give a colourless oil (0.304 g, 34%).

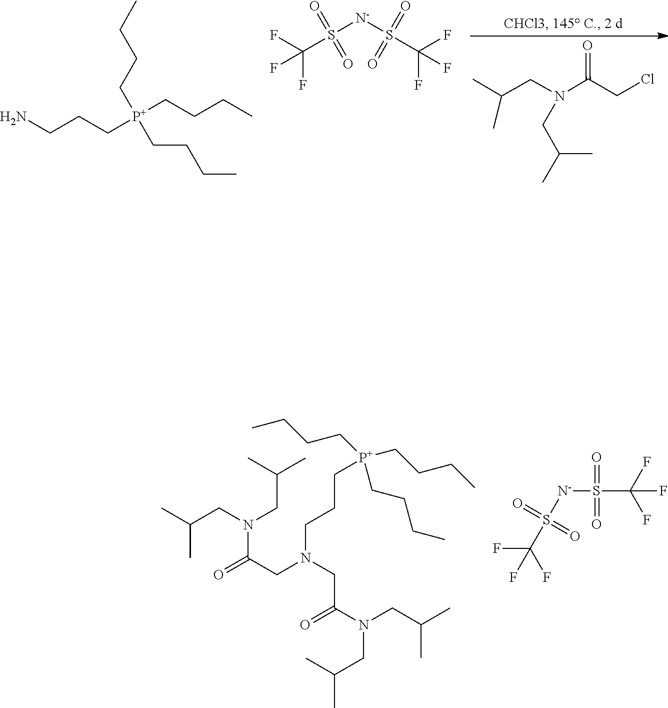 [SMILES: CCCC[P+](CCCC)(CCCC)CCCN ; O=S(=O)([N-]S(=O)(=O)C(F)(F)F)C(F)(F)F ; CC(C)CN(CC(C)C)C(=O)CCl ; CCCC[P+](CCCC)(CCCC)CCCN(CC(=O)N(CC(C)C)CC(C)C)CC(=O)N(CC(C)C)CC(C)C ; O=S(=O)([N-]S(=O)(=O)C(F)(F)F)C(F)(F)F]

To a high pressure vessel was added (3-Aminopropyl)(tributyl)phosphonium bistriflimide (0.200 g, 0.370 mmol), triethylamine (0.150 g, 1.48 mmol), N,N-diisobutyl-2-chloroacetamide (0.152 g, 0.740 mmol) and chloroform (5 mL). The vessel was stoppered and stirred at 145° C. on an oil bath for 48 hours. The reaction mixture was washed with pH 1 HCl (15 mL), then water (4×150 mL). The organic layer was dried over magnesium sulfate and concentrated in vacuo to give a viscous dark brown liquid, [MAIL-P₄₄₄⁺][NTf₂⁻], (0.250 g, 77%).

[MAIL-P₈₈₈⁺][NTf₂⁻]:

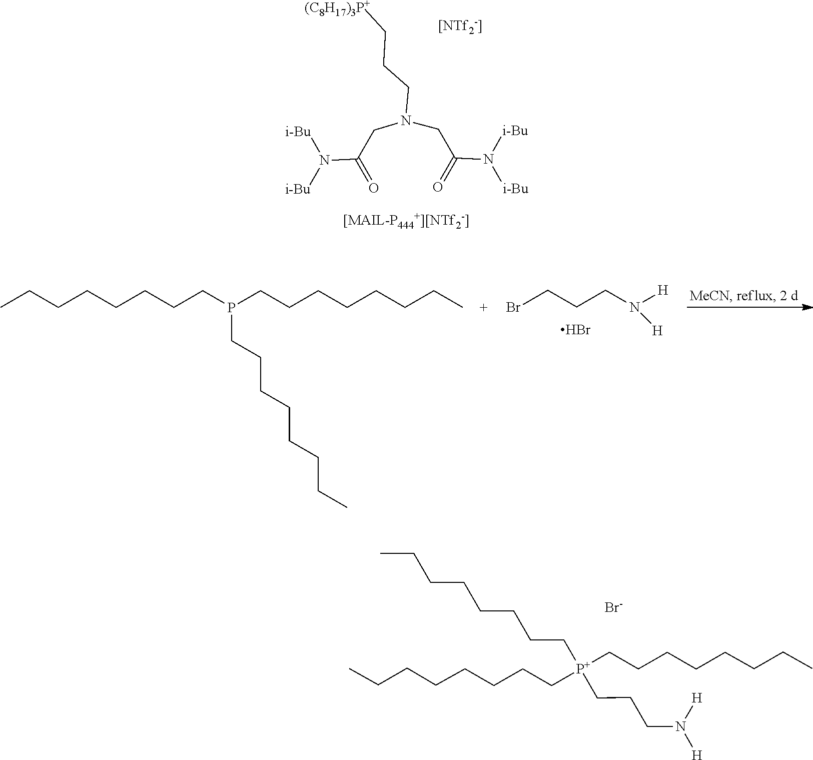

To a 50 mL round-bottom flask equipped with a magnetic stir bar trioctylphosphine (0.872 g, 2.35 mmol), 3-bromopropylamine hydrobromide (0.500 g, 2.28 mmol), and acetonitrile (25 mL) were added. The suspension was then heated and stirred at reflux for 48 hours. The reaction was cooled to room temperature, and the solvent was removed under reduced pressure, and the resulting oil was then dried in vacuo, and used in subsequent steps without further purification (0.889 g, 85%).

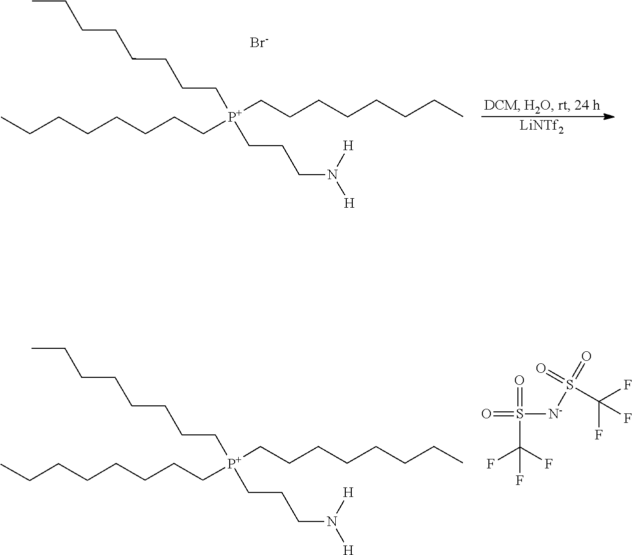

To a 50 mL round-bottom flask was added (3-Aminopropyl)(trioctyl)phosphonium bromide (0.564 g, 1.11 mmol) followed by DCM (20 mL). LiNTf₂ (0.954 g, 3.32 mmol) was added followed by deionised water (20 mL). The reaction mixture was stirred at room temperature for 24 hours. The aqueous layer was removed and the organic layer washed with deionised water (5×15 mL). The organic layer was dried over magnesium sulfate and concentrated. The product was dried overnight to give a colourless oil (0.542 g, 69%).

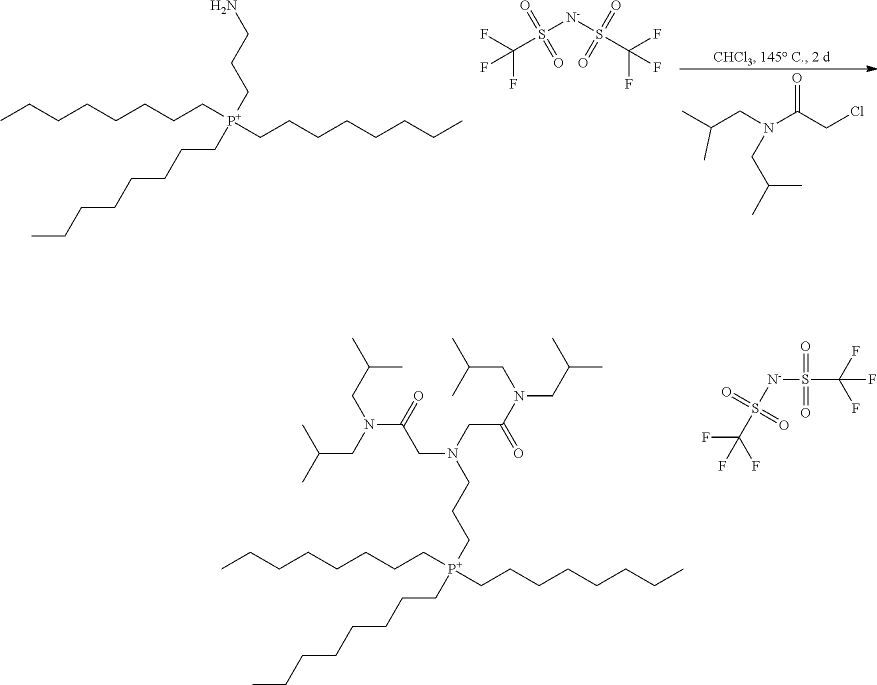 [SMILES: CCCCCCCC[P+](CCCN)(CCCCCCCC)CCCCCCCC ; O=S(=O)([N-]S(=O)(=O)C(F)(F)F)C(F)(F)F ; CC(C)CN(CC(C)C)C(=O)CCl ; CCCCCCCC[P+](CCCCCCCC)(CCCCCCCC)CCCN(CC(=O)N(CC(C)C)CC(C)C)CC(=O)N(CC(C)C)CC(C)C ; O=S(=O)([N-]S(=O)(=O)C(F)(F)F)C(F)(F)F]

To a high pressure vessel was added (3-Aminopropyl)(trioctyl)phosphonium bistriflimide (0.200 g, 0.282 mmol), triethylamine (0.114 g, 1.13 mmol), N,N-diisobutyl-2-chloroacetamide (0.116 g, 0.564 mmol) and chloroform (5 mL). The vessel was stoppered and stirred at 145° C. on an oil bath for 48 hours. The reaction mixture was washed with pH 1 HCl (15 mL), then water (4×150 mL). The organic layer was dried over magnesium sulfate and concentrated in vacuo to give a viscous dark brown liquid, [MAIL-P₈₈₈⁺][NTf₂⁻], (0.313 g, 99%).

### Example 2: Synthesis in Different Solvents

The [MAIL⁺] cation synthesis reaction described above was repeated using different solvents. The results are provided below:

| | Temperature | Time | Yield |
|---|---|---|---|
| Solvent | (° C.) | (hours) | (%) |
| Chloroform | 130 | 1 | 74 |
| Toluene | 130 | 1 | 51 |
| Dichloromethane | 130 | 1 | 43 |

It can be seen that good yields were obtained in just 1 hour when the reaction was carried out using different solvents, with particularly good results achieved in chloroform.

### Example 3: Liquid-Liquid Extraction of Rare Earth Metals Using [MAIL⁺][NTf₂⁻]

General Procedure for Extraction of Rare Earth Metals

Equal volumes (2 to 5 ml) of the ionic liquid extractant ([MAIL⁺][NTf₂⁻] in [P₆₆₆₍₁₄₎⁺][NTf₂⁻]) and an acidic aqueous feed solution containing rare earth metals in HCl were equilibrated for 15 to 30 minutes on a wrist action shaker. The phases were centrifuged and the aqueous phase was analysed for rare earth metal content using Inductively Coupled Plasma Optical Emission Spectroscopy (ICP-OES), though it will be appreciated that any suitable analysis technique may be used. The proportion of the rare earth metals extracted into the ionic liquid (organic) phase was determined through mass balance using the ICP-OES measurement.

The distribution ratio of an individual rare earth metal was determined as the ratio of its concentration in the ionic liquid phase to that of it in the aqueous phase (raffinate). D_{M}=[M]_{IL}/[M]_{Aq}, where IL represents ionic liquid phase and Aq represents the aqueous phase (raffinate).

The separation factor (SF) with respect to an individual rare earth metal pair is expressed as the ratio of the distribution ratio of a first rare earth metal with the distribution ratio of a second rare earth metal. For example, the separation factor of dysprosium with respect to neodymium=D_{Dy}/D_{Nd}. It will be appreciated that separation factors estimated from independently obtained distribution ratios will be lower than the actual separation factors, obtained during the separation of mixtures of rare earth metals during a competitive separation (as exemplified below).

Distribution ratios for individual rare earth metals were obtained in separate extractions according to the general procedure above, using 0.0075 M [MAIL⁺][NTf₂⁻] in [P₆₆₆₍₁₄₎⁺][NTf₂⁻] and a 200 mg/l (ppm) HCl solution of the relevant rare earth metal chloride (where 200 ppm refers to the concentration of the elemental metal in the solution). FIG. 1 shows a plot of the distribution ratios for each rare earth metal as a function of pH, showing that the ionic liquid according to the present invention may be used to extract rare earth metals across a range of pH values.

The separation of rare earth metals was also performed by the above method using 0.0075 M of the ionic liquids [MAIL⁺][R₂P(O)O⁻], [MAIL-6C⁺][NTf₂⁻] and [MAIL-Ph⁺][NTf₂⁻] in [P₆₆₆₍₁₄₎⁺][NTf₂⁻]. These ionic liquids were also found to differentially extract rare earth metals at pH 1 to pH4 as shown in FIGS. 3, 4 and 5.

Recycling of Ionic Liquid

Dy was extracted from an aqueous solution of Dy (180 ppm) at pH4 using 0.025 M [MAIL⁺][NTf₂⁻] in [P₆₆₆₍₁₄₎⁺][NTf₂⁻] (>95% extracted) and the ionic liquid stripped at pH 1 using HCl (1:1 ionic liquid to stripping solution ratio) in 4 contacts. The ionic liquid was washed with deionised water to raise the pH to 7, and was used in further extractions. The amount of Dy extracted dropped by around 20% compared to the first extraction, but remained at a constant level over four subsequent extractions.

Separation of Dy and Nd

An aqueous HCl solution containing DyCl₃.6H₂O (60 mg/l (ppm) Dy) and NdCl₃.6H₂O (1400 mg/l (ppm) Nd) at pH 3 was extracted with the ionic liquid extractant (0.005 M [MAIL⁺][NTf₂⁻] in [P₆₆₆₍₁₄₎⁺][NTf₂⁻]) according to the general procedure above. A single contact (extraction) gave D_{Dy}=13.45, D_{Nd}=0.0124, giving a SF_{Dy-Nd} of 1085.

This separation factor (1085) is considerably higher than the separation factors obtained for Dy/Nd separation by the systems in the prior art shown in Table 1 (maximum 239).

The above separation was repeated using 0.0075M of an ionic liquid in [P₆₆₆₍₁₄₎⁺][NTf₂⁻] at pH2. The extraction was performed using [MAIL⁺][NTf₂⁻], [MAIL⁺][R₂P(O)O⁻], [MAIL-6C⁺][NTf₂⁻], [MAIL-P₄₄₄⁺][NTf₂⁻], [MAIL-P₈₈₈⁺][NTf₂⁻], [MAIL-PPh₃⁺][NTf₂⁻] and [MAIL-PPh₃⁺][R₂P(O)O⁻] and the results are shown in Table 2. As can be seen, ionic liquids described herein can be used to completely selectively extract Dy from Nd. Completely selective extraction of Dy from Nd using [MAIL⁺][NTf₂⁻], [MAIL⁺][R₂P(O)O⁻]and [MAIL-6C⁺][NTf₂⁻] was also observed at pH 1.8, with extraction of more than 50% Dy.

TABLE 2

| | Dy % | Nd % |
|---|---|---|
| Ionic liquid | Extraction | Extraction |
| [MAIL⁺][NTf₂⁻] | 82 | 0 |
| [MAIL⁺][R₂P(O)O⁻] | 86.5 | 0 |
| [MAIL-6C⁺][NTf₂⁻] | 83 | 0 |
| [MAIL-P₄₄₄⁺][NTf₂⁻] | 89 | 0 |
| [MAIL-P₈₈₈⁺][NTf₂⁻] | 87 | 0 |
| [MAIL- PPh₃⁺][NTf₂⁻] | 90 | 0.6 |
| [MAIL-PPh₃⁺][R₂P(O)O⁻] | 90 | 0 |

Separation of Eu and La

An aqueous HCl solution containing EuCl₃.6H₂O (65 mg/l (ppm) Eu) and LaCl₃.7H₂O (470 mg/l (ppm) La) at pH 3 was extracted with the ionic liquid extractant (0.005 M [MAIL⁺][NTf₂⁻] in [P₆₆₆₍₁₄₎⁺][NTf₂⁻]) according to the general procedure above. A single contact (extraction) gave D_{Eu}=9.3, D_{La}=0.044, giving a SF_{Eu-La} of 211.

Separation of Tb and Ce

An aqueous HCl solution containing TbCl₃.6H₂O (530 mg/l (ppm) Tb) and CeCl₃.6H₂O (950 mg/l (ppm) Ce) at pH 3 was extracted with the ionic liquid extractant (0.0075 M [MAIL⁺][NTf₂⁻] in [P₆₆₆₍₁₄₎⁺][NTf₂⁻]) according to the general procedure above. A single contact (extraction) gave D_{Tb}=11.2, D_{Ce}=0.068, giving a SF_{Tb-Ce} of 162.

### Example 4: Stripping of Rare Earth Metals from [MAIL⁺][NTf₂⁻]

Dy(III) (80 ppm) was stripped from an organic phase at pH 0.25 comprising [MAIL⁺][NTf₂⁻] in [P₆₆₆₍₁₄)₊][NTf₂⁻] (0.0075 M) in 3 successive contacts. The organic phase was contacted with an equal volume of an aqueous HCl solution (0.55 M) and was equilibrated for 15 to 30 minutes on a wrist action shaker. 67 ppm of Dy(III) was stripped in the first contact, 10 ppm was stripped in the second contact, and 2 ppm was stripped in the third contact. Similarly, from observation of the distribution ratios in FIG. 1, it is clear that heavy rare earth metals such as Tm, Yb and Lu have significantly reduced distribution factors with increasing acidity. Thus, it is also expected that heavy rare earth metals may be stripped from the ionic liquid of the present invention at relatively high pH values.

The above examples show that a large increase in the separation factors between key rare earth metal pairs may be obtained by use of an ionic liquid according to the present invention (e.g. Nd/Dy: Nd-Dy magnet, Eu/La: white lamp phosphor, Tb/Ce: green lamp phosphor). The rare earth metals may also be advantageously stripped from the ionic liquid at relatively high pH compared to prior art systems.

Without wishing to be bound by any particular theory, it is believed that a more pronounced increase in distribution ratios is observed for heavier rare earth metals than lighter rare earth metals as a result of increased formation of the more hydrophobic doubly coordinated rare earth metal species M.([MAIL⁺][NTf₂⁻])₂ over the singly coordinated species M.([MAIL⁺NNTf₂⁻]). It is believed that the more hydrophobic species will be more easily extracted into the organic phase during separation, leading to increased distribution ratios.

Nuclear magnetic resonance, infra-red and mass spectrometry studies have shown that the doubly coordinated species is more abundant in solutions of Lu and the ionic liquid compared to solutions of La and the ionic liquid, highlighting the differentiation between the heavy and light rare earth metals achieved by the ionic liquid of the present invention.

Furthermore, optimised geometries of the complexes LaCl₃.([MAIL⁺][Cl⁻])₂ and LuCl₃.([MAIL⁺][Cl⁻])₂ show that the distance between the tertiary central nitrogen of the ionic liquid cation and the metal is much longer in the case of La (˜2.9 Å, non-bonding) than in the case of Lu (˜2.6 Å, bonding), which also supports the weaker bonding of the ionic liquid to lighter rare earth metals. At the same time, the electron donating groups, in this case amides, linked to the nitrogen atom bond to the metal in a very similar way in both cases. This result shows that the central motif of the ionic liquid cation having a tertiary nitrogen donor is important for the differentiation obtained between the heavier and lighter rare earth metals and the improved selectivity that results therefrom.

### Example 5: Extraction of Rare Earth Metals from a Magnet Sample

A magnet sample containing rare earth metals was obtained in powdered form and was converted to the chloride form as follows. The magnet feed was dissolved in 2 M H₂SO₄. The undissolved impurities were removed by filtration. The pH was raised to 1.5 using ammonium hydroxide at 60° C. At 60° C. the rare-earth sulphates crash out of solution leaving the iron sulphate impurity in solution. The separated rare-earth sulphate was converted to the oxalate (by contacting with oxalic acid to and washing the rare-earth oxalate with water) and calcined at 900° C. to form the rare-earth oxide. The rare-earth oxide is converted into the rare-earth chloride by leaching into a HCl solution and recrystallised.

A feed solution of 0.2 g rare-earth chloride salt in 50 mL pH 2 solution (HCl) was prepared. The feed solution had an initial concentration of 20.93 ppm Dy and 1573.81 ppm Nd.

Separate extractions were carried out as described in Example 2, using 0.0075 M [MAIL⁺][NTf₂⁻] or [MAIL⁺][R₂P(O)O⁻] in [P₆₆₆₍₁₄₎⁺][NTf₂⁻] at pH 2. The ionic liquids were both found to extract more than 90% of the Dy in the solution after 4 contacts, whilst extracting less than 5% Nd.

1. A process for preparing a cationic species [Cat⁺] for an ionic liquid, the cationic species having the structure: 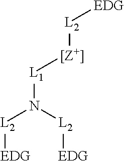
where: [Z⁺] represents a group selected from ammonium, benzimidazolium, benzofuranium, benzothiophenium, benzotriazolium, borolium, cinnolinium, diazabicyclodecenium, diazabicyclononenium, 1,4-diazabicyclo[2.2.2]octanium, diazabicyclo-undecenium, dithiazolium, furanium, guanidinium, imidazolium, indazolium, indolinium, indolium, morpholinium, oxaborolium, oxaphospholium, oxazinium, oxazolium, iso-oxazolium, oxothiazolium, phospholium, phosphonium, phthalazinium, piperazinium, piperidinium, pyranium, pyrazinium, pyrazolium, pyridazinium, pyridinium, pyrimidinium, pyrrolidinium, pyrrolium, quinazolinium, quinolinium, iso-quinolinium, quinoxalinium, quinuclidinium, selenazolium, sulfonium, tetrazolium, thiadiazolium, iso-thiadiazolium, thiazinium, thiazolium, iso-thiazolium, thiophenium, thiuronium, triazinium, triazolium, iso-triazolium and uronium groups;
L₁ represents a linking group selected from C₁₋₁₀ alkanediyl, C₂₋₁₀ alkenediyl, C₁₋₁₀ dialkanylether and C₁₋₁₀ dialkanylketone groups;
each L₂ represents a linking group independently selected from C₁₋₂ alkanediyl, C₂ alkenediyl, C₁₋₂ dialkanylether and C₁₋₂ dialkanylketone groups; and
each EDG represents an electron donating group;
said process comprising carrying out the following reaction: 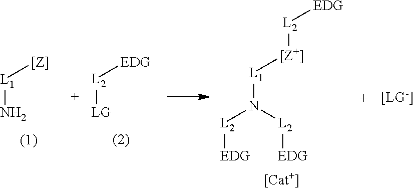
where: LG represents a leaving group;
wherein the process is carried out in a sealed reactor at a temperature of at least 100° C.

2. The process of claim 1, wherein the process is carried out at a temperature of from 100 to 180° C., preferably from 115 to 170° C., and more preferably from 125 to 145° C.

3. The process of claim 1 or claim 2, wherein the reaction is carried out for a period of from 0.5 to 24 hours, preferably from 1 to 12 hours and more preferably from 2 to 6 hours.

4. The process of any of claims 1 to 3, wherein the process is carried out at a pressure of from 105 to 500 kPa, preferably from 200 to 400 kPa, and more preferably from 250 to 350 kPa.

5. The process of any of claims 1 to 4, wherein reagent (2) is used in an amount of from 1 to 6 molar equivalents, preferably from 2 to 4 molar equivalents, and more preferably from 2.5 to 3.5 molar equivalents as compared to reagent (1).

6. The process of any of claims 1 to 5, wherein the reaction is carried out in the presence of a base, preferably a nitrogen-containing base, and more preferably a trialkylamine such as trimethylamine, the base preferably being used in an amount of from 1 to 10 molar equivalents, preferably from 2 to 8 molar equivalents, and more preferably from 3 to 5 molar equivalents as compared to reagent (1).

7. The process of any of claims 1 to 6, wherein the reaction is carried out in the presence of a protic solvent, such as trichloromethane.

8. The process of any of claims 1 to 7, wherein L₁ represents:
a linking group selected from C₁₋₁₀ alkanediyl and C₁₋₁₀ alkenediyl groups;
preferably a linking group selected from C₁₋₆ alkanediyl and C₂₋₅ alkenediyl groups;
more preferably a linking group selected from C₁₋₆ alkanediyl groups;
and still more preferably a linking group selected from —CH₂—, —C₂H₄— and —C₃H₆—.

9. The process of any of claims 1 to 8, wherein each L₂ represents:
a linking group independently selected from C₁₋₂ alkanediyl and C₂ alkenediyl groups;
preferably a linking group selected from C₁₋₂ alkanediyl groups;
and more preferably a linking group selected from —CH₂— and —C₂H₄—.

10. The process of any of claims 1 to 9, wherein each EDG represents:
an electron donating group independently selected from —CO₂R^{x}, —OC(O)R^{x}, —CS₂R^{x}, —SC(S)R^{x}, —S(O)OR^{x}, —OS(O)R^{x}, —NR^{x}C(O)N R^{y}R^{z}, —NR^{x}C(O)OR^{y}, —OC(O)NR^{y}R^{z}, —NR^{x}C(S)OR^{y}, —OC(S)NR^{y}R^{z}, —NR^{x}C(S)SR^{y}, —SC(S)NR^{y}R^{z}, —NR^{x}C(S)N R^{y}R^{z}, —C(O)NR^{y}R^{z}, —C(S)NR^{y}R^{z}, wherein R^{x}, R^{y} and R^{z} are independently selected from H or C₁₋₆ alkyl;
and preferably an electron donating group independently selected from —CO₂R^{x} and —C(O)NR^{y}R^{z}, wherein R^{x}, R^{y} and R^{z} are each independently selected from C₃₋₆ alkyl.

11. The process of claim 10, wherein each -L₂-EDG represents an electron donating group independently selected from: 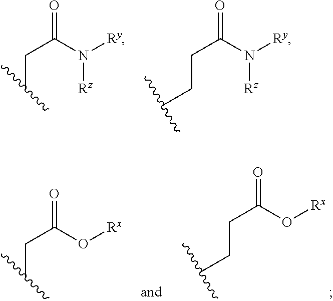
and preferably from: 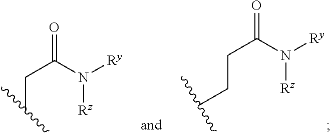
wherein R^{y}═R^{z}, and wherein R^{x}, R^{y} and R^{z} are each selected from C₃₋₆ alkyl, preferably C₄ alkyl, for example i-Bu.

12. The process of any of claims 1 to 11, wherein [Z⁺] represents:
an acyclic cation selected from:
[—N(R^{a})(R^{b})]⁺, [—P(R^{a})(R^{b})]⁺ and [—S(R^{a})]⁺,
where: R^{a} and R^{b} are each independently selected from optionally substituted C₁₋₃₀ alkyl, C₃₋₈ cycloalkyl and C₆₋₁₀ aryl groups.
or a cyclic cation selected from: 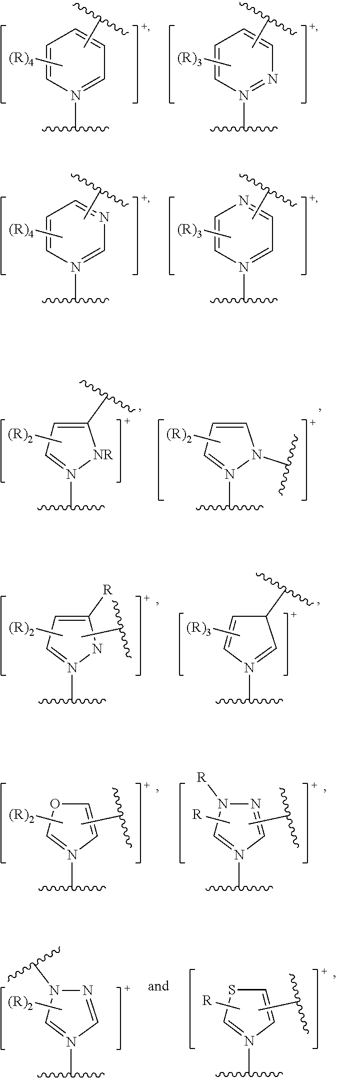
where: each R group is independently selected from: hydrogen and optionally substituted C₁₋₃₀ alkyl, C₃₋₈ cycloalkyl and C₆₋₁₀ aryl groups, or any two R groups attached to adjacent carbon atoms form an optionally substituted methylene chain —(CH₂)_{q}— where q is from 3 to 6;
or a saturated heterocyclic cation having the formula: 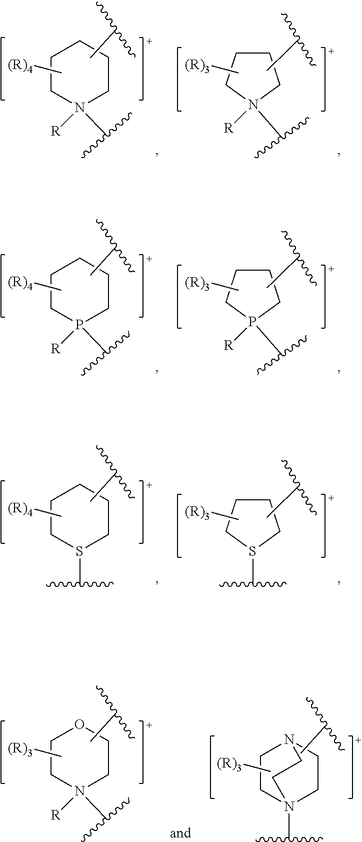
where: each R group is independently selected from: hydrogen and optionally substituted C₁₋₃₀ alkyl, C₃₋₈ cycloalkyl and C₆₋₁₀ aryl groups, or any two R groups attached to adjacent carbon atoms form an optionally substituted methylene chain —(CH₂)_{q}— where q is from 3 to 6.

13. The process of claim 12, wherein [Z⁺] represents a cyclic cation selected from: 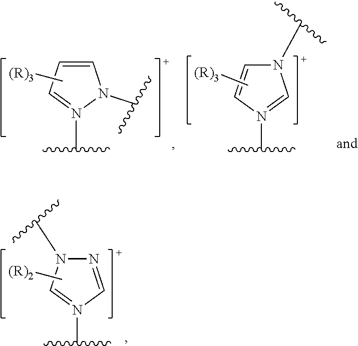
and preferably represents the cyclic cation:

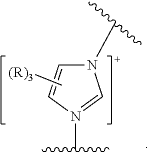

14. The process of any of claims 1 to 13, wherein -LG represents a leaving group selected from —OSO₂CF₃ (i.e. —OTf), —SO₂R such as tosylate (—OTs) or mesylate (—OMs), halides (such as —Cl, —Br and —I), —OR, —OR₂⁺, —ONO₂, —PO(OR)₂, —N₂⁺, —SR₂⁺, and —NR₃⁺, where R is selected from H, C₁₋₆ alkyl and C₄₋₁₀ aryl groups.

15. A process for preparing an ionic liquid having the formula [Cat⁺][X⁻], said process comprising:
preparing an ionic liquid having the formula [Cat⁺][LG³¹ ] using a process as defined in any of claims 1 to 14; and
where La is not the same as X⁻, carrying out the following reaction:
[Cat⁺][LG³¹ ]+[X⁻]→[Cat⁺][X⁻]+[LG⁻].

16. The process of claim 15, wherein [X⁻] is used in an amount of from 1 to 2.5 molar equivalents, preferably from 1.05 to 2 molar equivalents, and more preferably from 1.1 to 1.5 molar equivalents as compared to reagent (1).

17. The process of claim 15 or claim 16, wherein the reaction is carried out for a period of from 0.1 to 5 hours, preferably from 0.25 to 3 hours, and more preferably from 0.5 to 2 hours.

18. The process of any of claims 15 to 17, wherein the reaction is carried out in the presence of an organic solvent protic solvent, and preferably a halogenated solvent such as trichloromethane.

19. The process of any of claims 15 to 18, wherein the ionic liquid [Cat⁺][X−] is obtained at a yield of greater than 50%, preferably greater than 60%, and more preferably greater than 70%.

20. The process of any of claims 15 to 19, wherein [X⁻]represents one or more anionic species selected from:
hydroxides, halides, perhalides, pseudohalides, sulphates, sulphites, sulfonates, sulfonimides, phosphates, phosphites, phosphonates, phosphinates, methides, borates, carboxylates, azolates, carbonates, carbamates, thiophosphates, thiocarboxylates, thiocarbamates, thiocarbonates, xanthates, thiosulfonates, thiosulfates, nitrate, nitrite, tetrafluoroborate, hexafluorophosphate and perchlorate, halometallates, amino acids, borates, polyfluoroalkoxyaluminates;
preferably selected from:
bistriflimide, triflate, bis(alkyl)phosphinates such as bis(2,4,4-trimethylpentyl)phosphinate, tosylate, perchlorate, [Al(OC(CF₃)₃)₄⁻], tetrakis[3,5-bis(trifluoromethyl)phenyl]borate, tetrakis(pentafluorophenyl)-borate, tetrafluoroborate, hexfluoroantimonate and hexafluorophosphate anions;
and more preferably selected from:
bistriflimide, triflate and bis(2,4,4-trimethylpentyl)phosphinate anions.

21. A method for extracting a rare earth metal from a mixture of one or more rare earth metals, said method comprising:
preparing an ionic liquid using a process as defined in any of claims 15 to 20; and
contacting an acidic solution of the rare earth metal with a composition which comprises the ionic liquid to form an aqueous phase and a non-aqueous phase into which the rare earth metal has been selectively extracted.

22. The method of claim 21, wherein the method comprises recovering the rare earth metal from the non-aqueous phase, for instance by stripping with an acidic stripping solution, e.g. an aqueous hydrochloric acid or nitric acid solution, the acidic stripping solution preferably having a pH of 1 or lower and preferably a pH of 0 or higher.

23. The method of claim 21 or claim 22, wherein the acidic solution comprises a first and a second rare earth metal, and the method comprises:
(a) preferentially partitioning the first rare earth metal into the non-aqueous phase.

24. The method of claim 23, wherein the method further comprises, in step (a), separating the non-aqueous phase from the acidic solution; and
(b) contacting the acidic solution depleted of the first rare earth metal with the composition which comprises an ionic liquid, and optionally recovering the second rare earth metal therefrom;
and preferably wherein:
the first rare earth metal is recovered from the non-aqueous phase in step (a), and said non-aqueous phase is recycled and used as the composition in step (b); and/or
the acidic solution has a pH of less than 3.5 in step (a), and the acidic solution has a pH of greater than 3.5 in step (b).

25. The method of claim 23 or claim 24, wherein:
the first rare earth metal is dysprosium and the second rare earth metal is neodymium; or
the first rare earth metal is europium and the second rare earth metal is lanthanum.

26. The method of any of claims 21 to 25, wherein:
the acidic solution from which the rare earth metal is extracted has a pH of from 2 to 4;
the composition is added to the acidic solution in a volume ratio of from 0.5:1 to 2:1, preferably 0.7:1 to 1.5:1, more preferably 0.8:1 to 1.2:1, for example 1:1;
prior to contacting the composition with the acidic solution of the rare earth metal the composition is equilibrated with an acidic solution having the same pH as the acidic solution of the rare earth metal;
the acidic solution is contacted with the composition for from 1 to 40 minutes, preferably from 5 to 30 minutes; and/or
the method comprises contacting and physically mixing the acidic solution of the rare earth metal and the composition.

27. The method of any of claims 21 to 26, wherein the composition further comprises a lower viscosity ionic liquid and/or one or more organic solvents, and the ionic liquid is preferably present in the composition in a concentration of at least 0.001 M, preferably from 0.005 M to 0.01 M, for example 0.0075 M.

28. The method of any of claims 21 to 27, wherein the acidic solution is obtainable by leaching the rare earth metal from its source using an acid, the source of the rare earth metal preferably being a mineral or a waste material.
